SEQUENCE LISTING (30 sequences)

SEQ ID NO: 1 - protein, 1228 aa, Bacillus thuringiensis
MTSNRKNENEIINAVSNHSAQMDLLPDARIEDSLCIAEGNNIDPFVSASTVQTGINIAGRILGVLGVPFAGQLASFYSFLVGELWPRGRDQWEIFLEHVEQLINQQITENARNTALARLQGLGDSFRAYQQSLEDWLENRDDARTRSVLYTQYIALELDFLNAMPLFAIRNQEVPLLMVYAQAANLHLLLLRDASLFGSEFGLTSQEIQRYYERQVERTRDYSDYCVEWYNTGLNSLRGTNAASWVRYNQFRRDLTLGVLDLVALFPSYDTRTYPINTSAQLTREVYTDAIGATGVNMASMNWYNNNAPSFSAIEAAAIRSPHLLDFLEQLTIFSASSRWSNTRHMTYWRGRTIQSRPIGGGLNTSTHGATNTSINPVTLRFASRDVYRTESYAGVLLWGIYLEPIHGVPTVRFNFTNPQNISDRGTANYSQPYESPGLQLKDSETELPPETTERPNYESYSHRLSHIGIILQSRVNVPVYSWTHRSADRTNTIGPNRITQIPMVKASELPQGTTVVRGPGFTGGDILRRTNTGGFGPIRVTVNGPLTQRYRIGFRYASTVDFDFFVSRGGTTVNNFRFLRTMNSGDELKYGNFVRRAFTTPFTFTQIQDIIRTSIQGLSGNGEVYIDKIEIIPVTATFEAEYDLERAQEAVNALFTNTNPRRLKTDVTDYHIDQVSNLVACLSDEFCLDEKRELLEKVKYAKRLSDERNLLQDPNFTSINKQPDFISTNEQSNFTSIHEQSEHGWWGSENITIQEGNDVFKENYVTLPGTFNECYPTYLYQKIGESELKAYTRYQLRGYIEDSQDLEIYLIRYNAKHETLDVPGTESLWPLSVESPIGRCGEPNRCAPHFEWNPDLDCSCRDGEKCAHHSHHFSLDIDVGCTDLHENLGVWVVFKIKTQEGHARLGNLEFIEEKPLLGEALSRVKRAEKKWRDKREKLQLETKRVYTEAKEAVDALFVDSQYDRLQADTNIGMIHAADKLVHRIREAYLSELPVIPGVNAEIFEELEGHIITAISLYDARNVVKNGDFNNGLTCWNVKGHVDVQQSHHRSDLVIPEWEAEVSQAVRVCPGCGYILRVTAYKEGYGEGCVTIHEIENNTDELKFKNREEEEVYPTDTGTCNDYTAHQGTAGCADACNSRNAGYEDAYEVDTTASVNYKPTYEEETYTDVRRDNHCEYDRGYVNYPPVPAGYVTKELEYFPETDTVWIEIGETEGKFIVDSVELLLMEE

SEQ ID NO: 2 - protein, 1177 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWIRYNQFRRELTLTVLDIVSLFPNYDSRTYPIRTVSQLTREIYTNPVLENFDGSFRGSAQGIEGSIRSPHLMDILSSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKQEVSEKVKHAKRLSDERNLLQDPNFRGINRQLDRGWRGSTDITIQGGDDVFKENYVTLPGTFDECYPTYLYQKIDESKLKAYTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGCIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEIYPNNTVTCNDYTVNQEEYEGAYTSRNRGYNEAPSVPADYASVYEEKSYTDGRRENPCEFNRGYRDYTLLPVGYVTKELEYFPETDKVWIEIGETEGTFIVDSVELLLMEE

SEQ ID NO: 3 - protein, 1277 aa, Bacillus thuringiensis
MLEPKELEGNHSKNVRITICLYNSMKKLFNGEKLNSCNVSTPKKGGVILTSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNEEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINTSAQLTREIYTDPIGRTNAPSGFASTNWFNNNAPSFSAIEAAIFRPPHLLDFPEQLTIYSASSRWSSTQHMNYWVGHRLNFRPIGGTLNTSTQGLTNNTSINPVTLQFTSRDVYRTESNAGTNILFTTPVNGVPWARFNFINPQNIYERGATTYSQPYQGVGIQLFDSETELPPETTERPNYESYSHRLSHIGLIIGNTLRAPVYSWTHRSADRTNTIGPNRITQIPLVKALNLHSGVTVVGGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQKAVNALFTSTNPRRLKTDVTDYHIDQVSNMVACLSDEFCLDEKRELFEKVKYAKRLSDERNLLQDPNFTFISGQLSFASIDGQSNFPSINELSEHGWWGSANVTIQEGNDVFKENYVTLPGTFNECYPNYLYQKIGESELKAYTRYQLRGYIEDSQDLEIYLIRYNAKHETLDVPGTDSLWPLSVESPIGRCGEPNRCAPHFEWNPDLDCSCRDGERCAHHSHHFTLDIDVGCTDLHENLGVWVVFKIKTQEGYARLGNLEFIEEKPLIGEALSRVKRAEKKWRDKREKLQLETKRVYTEAKEAVDALFVDSQYDQLQADTNIGMIHAADKLVHRIREAYLSELPVIPGVNAEIFEELEGHIITAMSLYDARNVVKNGDFNNGLTCWNVKGHVDVQQSHHRSDLVIPEWEAEVSQAVRVCPGRGYILRVTAYKEGYGEGCVTIHEIENNTDELKFKNCEEEEVYPTDTGTCNDYTAHQGTAACNSRNAGYEDAYEVDTTASVNYKPTYEEETYTDVRRDNHCEYDRGYVNYPPVPAGYVTKELEYFPETDTVWIEIGETEGKFIVDSVELLLMEE

SEQ ID NO: 4 - protein, 1167 aa, Bacillus thuringiensis
MEINNQKQCIPYNCLSNPEEVLLDGERILPDIDPLEVSLSLLQFLLNNFVPGGGFISGLVDKIWGALRPSEWDLFLAQIERLIDQRIEATVRAKAITELEGLGRNYQIYAEAFKEWESDPDNEAAKSRVIDRFRILDGLIEANIPSFRIIGFEVPLLSVYVQAANLHLALLRDSVIFGERWGLTTKNVNDIYNRQIREIHEYSNHCVDTYNTELERLGFRSIAQWRIYNQFRRELTLTVLDIVALFPNYDSRLYPIQTFSQLTREIVTSPVSEFYYGVINSGNIIGTLTEQQIRRPHLMDFFNSMIMYTSDNRREHYWSGLEMTAYFTGFAGAQVSFPLVGTRGESAPPLTVRSVNDGIYRILSAPFYSAPFLGTIVLGSRGEKFDFALNNISPPPSTIYRHPGTVDSLVSIPPQDNSVPPHRGSSHRLSHVTMRASSPIFHWTHRSATTTNTINPNAIIQIPLVKAFNLHSGATVVRGPGFTGGDILRRTNTGTFADMRVNITGPLSQRYRVRIRYASTTDLQFFTRINGTTVNIGNFTRTMNSGDNLESGNFRTAGFSTPFSFSNAQSTFTLGTQPFSNQEVYIDRIEFVPAEVTFEAESDLERAQKAVNALFTSTNQLGLKTDVTDYQIDQVSNLVECLSDEFCLDEKRELSEKVKHAKRLSDKRNLLQDPNFTSINRQLDRGWRGSTDITIQGGNDVFKENYVTLPGTFDECYPTYLYQKIDESKLKAYTRYELRGYIEDSQDLEVYLIRYNAKHETVNVPGTGSLWPLSVESPIGRCGEPNRCVPHIEWNPDLDCSCRDGEKCAHHSHHFSLDIDVGCTDLNEDLGVWVIFKIKTQDGHARLGNLEFLEEKPLLGEALARVKRAEKKWRDKREQLQFETNIVYKEAKESVDALFVDSHYNRLQADTNITMIHAADKRVHRIREAYLPELSVIPGVNADIFEELEGLIFTAFSLYDARNIIKNGDFNNGLSCWNVKGHVDIQQNDHRSVLVVPEWESEVSQEVRVCPGRGYILRVTAYKEGYGEGCVTIHEIEDNTDELKFSNCIEEEVYPTDTGNDYTAHQGTTGCADACNSRNVGYEDGYEINTTASVNYKPTYEEEMYTDVRRDNHCEYDRGYGNHTPLPAGYVTKELEYFPETDTVWIEIGETEGTFIVDSVELLLMEE

SEQ ID NO: 5 - protein, 7 aa, Artificial Sequence
  PhD peptide
SKHSLSQ

SEQ ID NO: 6 - protein, 7 aa, Artificial Sequence
  PhD peptide
TTKLPNS

SEQ ID NO: 7 - protein, 7 aa, Artificial Sequence
  PhD peptide
ETPSRAR

SEQ ID NO: 8 - protein, 7 aa, Artificial Sequence
  PhD peptide
NNSGKQL

SEQ ID NO: 9 - protein, 20 aa, Bacillus thuringiensis
EDSLCIAEGNNIDPFVSAST

SEQ ID NO: 10 - protein, 8 aa, Bacillus thuringiensis
EIIPVTAT

SEQ ID NO: 11 - DNA, 12 bp, Bacillus thuringiensis
gaggatagcttg

SEQ ID NO: 12 - DNA, 12 bp, Bacillus thuringiensis
gaaattattcca

SEQ ID NO: 13 - DNA, 20 bp, Artificial Sequence
  Primer
ccctcatagttagcgtaacg

SEQ ID NO: 14 - protein, 6 aa, Bacillus thuringiensis
VLGVPF

SEQ ID NO: 15 - protein, 9 aa, Bacillus thuringiensis
GVLLWGIYL

SEQ ID NO: 16 - protein, 7 aa, Artificial Sequence
  PhD peptide
NRTVPNL

SEQ ID NO: 17 - protein, 7 aa, Artificial Sequence
  PhD peptide
KGPLPGQ

SEQ ID NO: 18 - protein, 7 aa, Artificial Sequence
  PhD peptide
MRFPGDL

SEQ ID NO: 19 - protein, 7 aa, Artificial Sequence
  PhD peptide
SHPHLKT

SEQ ID NO: 20 - protein, 7 aa, Artificial Sequence
  PhD peptide
NIKSSHV

SEQ ID NO: 21 - protein, 7 aa, Artificial Sequence
  PhD peptide
ITRTSHT

SEQ ID NO: 22 - protein, 7 aa, Artificial Sequence
  PhD peptide
MSVSTRD

SEQ ID NO: 23 - protein, 7 aa, Artificial Sequence
  PhD peptide
KTSLHHM

SEQ ID NO: 24 - protein, 7 aa, Artificial Sequence
  PhD peptide
SMLDKSQ

SEQ ID NO: 25 - DNA, 67 bp, Artificial Sequence
  Primer
tttgaattcagtaagcattctctttctcagggggggtcgggggggtcgatggtgagcaagggcgagg

SEQ ID NO: 26 - DNA, 67 bp, Artificial Sequence
  Primer
tttgaattcacgacgaagttgcctaattcgggggggtcgggggggtcgatggtgagcaagggcgagg

SEQ ID NO: 27 - DNA, 67 bp, Artificial Sequence
  Primer
tttgaattcgagacgccgtctcgggcgaggggggggtcgggggggtcgatggtgagcaagggcgagg

SEQ ID NO: 28 - DNA, 67 bp, Artificial Sequence
  Primer
tttgaattcaataatagtgggaagcagcttggggggtcgggggggtcgatggtgagcaagggcgagg

SEQ ID NO: 29 - DNA, 36 bp, Artificial Sequence
  Primer
gcttggctgaagcttcttgtacagctcgtccatgcc

SEQ ID NO: 30 - DNA, 18 bp, Artificial Sequence
  Linker
ggggggtcgggggggtcg

(19) United States
(12) Patent Application Publication
(10) Pub. No.: US 2018/0127771 A1
(43) Pub. Date: May 10, 2018

(54) INSECTICIDAL TOXINS FOR PLANT RESISTANCE TO HEMIPTERA

(71) Applicants: Iowa State University Research Foundation, Inc., Ames, IA (US); United States of America, as represented by the Secretary of Agriculture, Washington, DC (US)

(72) Inventors: BRYONY C. BONNING, Gainsville, FL (US); NANASAHEB CHOUGULE, AMES, IA (US); MARIA TERESA FERNANDEZ-LUNA, AMES, IA (US); MICHAEL B. BLACKBURN, BELTSVILLE, MD (US); DAVID G. HALL, FORT PIERCE, FL (US)

(21) Appl. No.: 15/808,706

(22) Filed: Nov. 9, 2017

Related U.S. Application Data
(60) Provisional application No. 62/420,078, filed on Nov. 10, 2016.

(51) Int. Cl. C12N 15/82; A01M 1/20; C12N 5/14

(52) U.S. Cl. CPC C12N 15/8286; A01N 63/00; C12N 5/14; A01M 1/20

(57) ABSTRACT

Provided are chimeric Hemiptera-active insecticidal toxin proteins comprising a Bt toxin peptide with activity against Hemiptera, a peptide multimer or fusion protein containing such peptide which binds to the gut of sap-sucking insects, preferably the Asian citrus psyllid, Diaphorina citri. This insect carries a bacterium associated with Huanlongbing disease or citrus greening and damages citrus crops. When bound, this peptide mediates the binding of the chimeric insecticidal protein to the target insect gut. Also described are coding sequences, vectors, and transgenic plants genetically modified to contain and express such insecticidal proteins. Other delivery mechanisms such as use of phloem-inhabiting bacteria, are also contemplated. Thus, the use of these toxins reduces economic loss due to feeding by the target insect and also reduces loss due to plant diseases spread by the target insect.

### CROSS REFERENCE TO RELATED APPLICATION

The present application claims priority to the earlier filed U.S. Provisional Application having Ser. No. 62/420,078, and hereby incorporates subject matter of the provisional application in its entirety.

### GRANT REFERENCE

This invention was made with government support under the U.S. Department of Agriculture research grant CRDF-120917. The government has certain rights in the invention.

### FIELD OF THE INVENTION

The invention relates generally to the field of molecular biology and plant genetics.

### BACKGROUND OF THE INVENTION

Sap-sucking insects (Hemiptera), including aphids and plant bugs, currently present one of the biggest challenges for insect pest management in United States agriculture. Management of hemipteran pests relies primarily on the application of environmentally damaging chemical insecticides.

The sap-sucking Asian citrus psyllid (ACP), Diaphorina citri Kuwayama (Hemiptera: Psyllidae), is one of the most serious pests of the U.S. and global citriculture. ACP transmits a pathogenic bacterium that causes huanglongbing (HLB) disease, which causes stunting, off-season bloom, premature fruit drop, increased fruit bitterness, tree decline, and eventually tree death. Severe economic losses are attributed to HLB world citriculture (Gottwald et al., (2007) http://www.plantmanagementnetwork.org/sub/php/review/2007/huanglongbing; Halbert and Manjunath, (2004) Fla Entomol, 87:330-353; Manjunath et al., (2008) Phytopathology, 98:287-296). There is no known cure for citrus HLB. Intensive insecticide programs against the ACP are currently advocated for preventing HLB in citrus (Bove, (2006) J Plant Pathol, 88:7-37). Such insecticide programs are expensive and environmentally disruptive, particularly with respect to non-target organisms. Moreover, insecticides commonly lose efficacy with the development of insecticide resistance, as most notably in ACP (Tiwari et al., (2012) Pest Manag Sci, 68: 1405-1412; Tiwari et al., (2012) J Econ Entomol, 105: 540-548) and other Hemiptera such as aphids and whiteflies (Devonshire, (1989) Pest Manag Sci, 26:375-382, ; Foster et al., (2002) Pest Manag Sci, 58:895-907). Taking advantage of a more sustainable approach, toxins derived from the bacterium, Bacillus thuringiensis (Bt), have been used successfully for management of other insect pests (Christou et al., (2006) Trends Plant Sci, 11:302-308; Shelton et al., (2002) Annual Review of Entomology, 47:845-881).

The use of transgenic crops expressing insecticidal proteins from Bt has become a primary approach for lepidopteran and coleopteran pest management (Shelton et al., 2002, Christou et al., 2006). The use of Bt transgenic crops has benefited both the growers through crop protection and the environment by reducing the use of environmentally damaging, classical chemical insecticides (Shelton et al., 2002). Hemipteran pests with piercing and sucking mouthparts are not particularly susceptible to the effects of Bt toxins, which may result from lack of exposure to B. thuringiensis, which exists in the soil and on the surface of foliage. Hence, there has been no natural selection for toxicity of Bt toxins to the Hemiptera (Schnepf et al., (1998) Microbiol Mol Biol R, 62: 775-806). Indeed, the deleterious impact of aphids and plant bugs on Bt cotton and Bt corn is increasing, thereby compromising the success of the Bt technology because of the need to apply chemical insecticides for hemipteran pests (Greene et al., (1999) J Econ Entomol, 92:941-944; Greene et al., (2001) J Econ Entomol, 94:403-409).

Transgenic crops expressing Bt toxins play a primary role for management of lepidopteran (moth) and coleopteran (beetle) pests. Several reports have demonstrated low levels of toxicity to aphids at high Bt toxin doses: Feeding assays indicated some toxicity of the Bt toxins Cry2, Cry3A, and Cry4 against the potato aphid, Macrosiphum euphorbiae (Walters & English, (1995) Entomol Exp Appl, 77:211-216, 1995, Walters et al., (1994) Insect Biochem Mol Biol, 24:963-968). Porcar et al (2009) demonstrated low to moderate toxicity of Cry3A, Cry4Aa and Cry11Aa to the pea aphid with 100% mortality following feeding on 125 to 500 μg/mL Cry4 or Cry11 (Porcar et al., (2009) Appl Envoron Microbiol, 75: 4897-4900). For comparison with known toxicity against species that are susceptible to Cry toxins, the LC50 of Cry1Ac against Heliothis virescens is around 1 μg/mL (Ali et al., (2006) J Econ Entomol, 99: 164-175), and the LC50 of Cry3Aa against Leptinotarsa decemlineata is 3.56 μg/mL (Park et al., (2009) Appl Enciron Microbiol, 75:3086-3092). Analyses of the impact of transgenic plants expressing Cry toxins on aphids gave variable results ranging from minor negative effects on aphid survival and fecundity to significant beneficial effects on aphid populations (Ashouri, (2004) Commun Agric Appl Biol Sci, 69:273-280; Ashouri et al., (2004) Comun Agric Appl Biol Sci, 69:205-209; Ashouri et al., (2001) Environ Entomol, 30:524-532; Faria et al., (2007) PLOS One, https://doi.org/10.1371/journal.pone.0000600; Mellet & Shoeman, (2007) Indian J Exp Biol, 45:554-562; Raps et al., (2001) Mol Ecol, 10:525-533; Lawo et al., (2009) PLOS One, https://doi.org/10.1371/journal.pone.0004804; Burgio et al., (2007) Bull Entomol Res, 97: 211-215).

The physiological basis for the low aphicidal toxicity of two representative Cry toxins has been examined for Cry1Ac and Cry3Aa in the pea aphid gut (Li et al., (2011) J Invert Pahtol, 107: 69-78). The interaction with the aphid varies with the toxin: Cry1Ac was processed by aphid gut proteases to produce active toxin, which bound to the aphid gut epithelium but showed low aphicidal activity. Cry3Aa was incompletely processed and partially degraded in the aphid gut resulting in production of low amounts of active toxin. Active Cry3Aa bound to brush border membrane vesicles (BBMV) proteins and showed a similar low level of toxicity against the pea aphid in bioassays. The mechanisms of Cry toxin action in aphids downstream of toxin binding remain to be explored.

There is a long felt need in the art for environmentally friendly and economical methods for reducing damage to both crop and ornamental plants from infestation with hemipteran pests. The present invention meets this need.

### SUMMARY OF THE INVENTION

Applicants have identified strains of Bt that produce toxins with insecticidal activity against hemipteran species and have also identified the specific toxins they produce. An embodiment of the invention is a novel insecticide composition effective against Hemiptera such as the Asian citrus psyllid, comprising an effective amount of a toxin produced by Bt strain IBL 00200, IBL-00068, IBL-00365, IBL-000681, IBL-00048, and/or IBL-00829. Another embodiment the invention includes an insecticidal composition effective against Hemiptera comprising a Cry1B protein or a fragment, derivative or conservative variant thereof with Hemiptera insecticidal activity. In yet another embodiment the invention includes an insecticidal composition which include a peptide having SEQ ID NO: 1 or SEQ ID NO: 2 and their conservatively modified variants.

Compositions of the invention will include the insecticidal proteins identified herein in an effective amount to kill or otherwise inhibit Hemiptera. The effective amount of the composition would comprise a peptide with 90% amino acid sequence identity or greater with SEQ. ID. NO: 1 or 2. It is contemplated that such a composition is applied to the environment of the hemipteran pests, within a food supply such as phloem. The composition may be formulated for preventive or prophylactic application to an area to prevent infestation of pests by introducing an alternative food source composition or trap plant comprising the toxins.

In another embodiment, a method for inhibiting hemipteran insect pests is included herewith, the method comprising selecting Bacillus thuringiensis Cry1Ba (SEQ ID NO: 1), Cry1Ab (SEQ ID NO: 2) or toxin produced by Bt isolate IBL-00200, IBL-00068, IBL-00365, IBL-00681, IBL-00048, IBL-00937, IBL-01306, IBL-01313, IBL-03792 and/or IBL-00829, and applying an effective amount of said protein to the insect pest, or to an available food source wherein the mortality of said insect increases.

In a particularly preferred embodiment the Hemipteran toxins are modified to create chimeric proteins designed for improved uptake by Hemiptera and other sap-sucking insects. Here is provided a modified insecticidal toxin that specifically binds to a receptor in a sap-sucking insect gut, especially an Asian citrus psyllid, via a peptide or peptide multimers incorporated within the modified insecticidal toxin. For example, the toxins could be modified by an N-terminal extension or by incorporation within surface domains of the toxin. By mediating the gut binding of the insecticidal protein, the toxin is acquired by the insect feeding on a plant which expresses the chimeric toxin or to which the chimeric toxin has been introduced, or to a food source composition comprising the toxin, and plant pests feeding on the composition or plant are killed. The use of this modified toxin disclosed herein applies to primarily to insects which feed on plant fluids (sap-sucking insects), including but not limited to, aphids and planthoppers, whiteflies (Hemiptera) and most particularly the Asian citrus psyllid, Diaphorina citri.

According to the invention, gut binding peptides may be incorporated into multiple loops of the Bt toxin to expand the range of target insects, and to increase uptake of the ingested toxin. Alternatively, one or more gut-binding peptides can be substituted in place of certain surface loops of an insecticidal protein, including but not limited to a Bt protein.

In a particular embodiment, the gut binding peptide portion comprises an amino acid sequence set forth in peptide 12 (SEQ ID NO: 5) (abbreviated as either pept12 or pept-12 throughout), peptide 15 (SEQ ID NO: 6) (abbreviated as either pept15 or pept-15 throughout), peptide 18 (SEQ ID NO: 7) (abbreviated as either pept18 or pept-18 throughout), or peptide 22 (SEQ ID NO: 8) (abbreviated as either pept22 or pept-22 throughout). An exemplary toxin component is that of Cry1Ab (SEQ ID NO: 2) or Cry1Ba (SEQ ID NO: 1) or those produced by strains IBL-00200, IBL-00068, IBL-00365, IBL-00681, IBL-00048, IBL-00937, IBL-01306, IBL-01313, IBL-03792 and/or IBL-00829 of B. thuringiensis (see sequences herein). The modified toxins are ingested by insects including Hemiptera or Asian citrus psyllid into the gut of the insect.

The invention further comprises providing a peptide or peptide multimer as part of the modified toxin that includes an amino acid sequence of a gut binding peptide, and bringing a source of food containing the modified insecticidal toxin into contact with the insect under conditions that allow the insect to ingest the food, whereby the modified toxin binds the gut epithelium and insect death ensues. In a preferred embodiment the food source is transgenic plant tissue, namely phloem. A phloem specific promoter may be used in this regard. As a result, feeding is reduced, and transmission of pathogens carried by the insect to a susceptible plant is reduced, and incidence and/or severity of disease caused by the pathogen is reduced. The peptides and peptide components of the modified insecticidal toxins specifically exemplified herein enable the inhibition of the spread of plant pathogens carried by certain Hemipteran plant pests, such the bacteria responsible for huanglongbing, (HLB, Citrus Greening) such as Candidatus Liberibacter africanus, Candidatus Liberibacter asiaticus and Candidatus Liberibacter americanus.

Gut binding peptides provided herein may be identified by standard phage display assays with ground animal gut proteins and those identified herein for ACP, which include the following: Peptide 12 (S K H S L S Q) (SEQ ID NO: 5); Peptide 15 (T T K L P N S) (SEQ ID NO: 6); Peptide 18 (E T P S R A R) (SEQ ID NO: 7); and/or Peptide 22 (N N S G K Q L) (SEQ ID NO: 8). Modifications of the toxins include the N-terminal and C-terminal regions of the Cry1Ba toxic core. The N-terminal sequencing results shows EDSLCIAEGNNIDPFVSAST (SEQ ID NO: 9) amino acid sequence while the C-terminus corresponds to EIIPVTAT (SEQ ID NO: 10) amino acid sequence. The protein fragment corresponding to the region composed by these boundaries results in a 608-residue sequence and is estimated for a 68.5 kDa. The corresponding 12 nucleotide sequence for the N terminus is 91 GAG GAT AGC TTG 102 (SEQ ID NO: 11). While the corresponding 12 nucleotide sequence for the C terminus is 1891 GAA ATT ATT CCA 1902 (SEQ ID NO: 12).

A nucleotide sequence encoding an amino acid sequence corresponding to the sequences described and disclosed herein can be incorporated into the coding sequence of a protein to form a fusion protein, especially where it is incorporated at the N-terminus of the protein (preceded by a Met residue to initiate transcription or where the recited sequence of amino acids is preceded by a signal peptide to allow secretion of the gut-binding fusion protein into the sap of a transgenic plant expressing same). Alternatively, such a peptide can be expressed as a peptide multimer (of identical peptide or one or more peptides of sequences).

In yet another embodiment plants susceptible to attack by sap-sucking insects are transformed to express a chimeric toxin as described herein in the fluids of the plant phloem, or xylem, upon which insects such as pysllids and aphids feed. A hemipteran insect feeding on the phloem of a transgenic plant expressing a chimeric toxin provided herein thereby acquires the chimeric toxin in its gut, where the peptide mediates gut binding of the chimeric toxin, such that the insect is killed or at least inhibited from feeding, thus providing for at least partial protection of the plant form the relevant plant pest and methods of insect control. Transgenic plants expressing the chimeric toxin including this peptide inhibit aphid feeding on the same and on other plants, lower the incidence of crop damage due to feeding by aphids and other sap-sucking insects and also lower virus infection in a crop of such plants where the virus is aphid transmitted thereby reducing damage in the crop as a whole. For other insects which feed on plant tissue, modified insecticidal toxin expression is directed to the appropriate tissue, such as leaf, stem or xylem or constitutive expression of the modified insecticidal toxin throughout the plant can be affected.

Further provided is a method of making a modified insecticidal toxin comprising a gut binding peptide portion and a toxin portion, said method comprising the step of identifying a gut binding peptide for a target insect which is not susceptible to naturally occurring Bt by panning a gut binding peptide from a peptide library using target insect gut epithelial tissue or target insect brush border membrane vesicles (BBMV) or target insect receptor protein, and fusing the nucleotide sequence encoding the gut binding peptide in frame with a Bt coding sequence, advantageously as an addition to the external loop of the toxin. Applicants have incorporated peptide 12 (SEQ ID NO: 5), peptide 15 (SEQ ID NO: 6), peptide 18 (SEQ ID NO: 7), or peptide 22 (SEQ ID NO: 8) into Cry1Ba (SEQ ID NO: 1) or Cry1Ab (SEQ ID NO: 2). Insertions or substitutions of a peptide or peptide multimer which binds to the gut membrane of an insect plant pest of interest can be made to render the chimeric toxin effective against that insect pest of the plant. Then stable introduction of the plant-expressible chimeric toxin gene into the plant and expression of that chimeric toxin gene in the plant allows for at least partial protection of the plant from the targeted insect pest.

In some embodiments, the present disclosure relates to a plant (such as a trap plant or the citrus plant itself (e.g., orange and/or grapefruit)) comprising an expression vector. A citrus plant or trap plant may comprise an expression vector in a single cell, a plurality of cells (e.g., mosaic), or in all cells. A mosaic plant may arise from a graft in some embodiments. For example, a citrus plant or trap plant may comprise a graft of a transgenic plant having an expression vector in all cells (e.g., scion) and a plant having a different expression vector or no expression vector in its cells (e.g., rootstock). A citrus plant or trap plant may comprise, in some embodiments, in a single cell, a plurality of cells (e.g., mosaic), or in all cells an expression vector (e.g., encoding hemipteran chimeric insecticidal peptide). For example, a citrus plant or trap plant cell may comprise (a) an expression vector, comprising, in a 5′ to 3′ direction, (i) an expression control sequence; (ii) a heterologous nucleic acid operably linked to the expression control sequence; and (iii) a 3′ termination sequence operably linked to the exogenous nucleic acid, wherein the first exogenous nucleic acid comprises a nucleic acid sequence encoding a protein having at least about 98% identity to a chimeric insecticidal protein, or a Cry1Ba (SEQ ID NO: 1) protein or a Cry1Ab (SEQ ID NO: 2) protein as disclosed herein.

The present disclosure relates, in some embodiments, to methods of expressing in a citrus plant or trap plant a heterologous nucleic acid comprising a nucleic acid sequence encoding an insecticidal peptide (e.g., a Bt toxin or chimeric Bt toxin with a gut binding protein). For example, a method may comprise contacting an expression cassette comprising an exogenous nucleic acid or an expression vector comprising an exogenous nucleic acid with the cytosol of a cell of a citrus plant or trap plant under conditions that permit expression of the exogenous nucleic acid and formation of the expressed peptide. In some embodiments, an expression vector and/or an expression cassette may comprise, in a 5′ to 3′ direction, an expression control sequence, the exogenous nucleic acid operably linked to the expression control sequence, and a 3′ termination sequence operably linked to the exogenous nucleic acid. An expressed peptide may comprise an amino acid sequence having at least 99% identity to an amino acid sequence selected from a Bt Cry1Ab (SEQ ID NO: 2) toxin, a Cry1Ba (SEQ ID NO: 1) toxin or a chimeric Bt toxin as disclosed herein. Contacting an expression vector or cassette may further comprise, in some embodiments, co-cultivating the cell with an Agrobacterium cell comprising the expression vector or expression cassette to form a co-cultivated plant cell. According to some embodiments, a plant may be regenerated from a co-cultivated plant cell.

The present disclosure relates, in some embodiments, to methods for treating a citrus plant having and/or at risk of having a microbial infection (e.g., citrus huanglongbing (ex. greening)). For example, a method may comprise forming in the citrus plant at least one insecticidal peptide. Forming in the citrus plant at least one insecticidal peptide may comprise, in some embodiments, grafting the citrus plant with a cutting (e.g., a scion or a rootstock) from a second citrus plant, the second citrus plant comprising an expression vector and/or an expression cassette comprising, in a 5′ to 3′ direction, an expression control sequence, an insecticidal nucleic acid operably linked to the expression control sequence, and a 3′ termination sequence operably linked to the insecticidal nucleic acid, wherein the insecticidal nucleic acid comprises a nucleic acid sequence encoding an amino acid sequence having at least 99% identity to an amino acid sequence as disclosed herein includes a toxin from IBL-00200, IBL-00068, IBL-00365, IBL-00681, IBL-00048, IBL-00937, IBL-01306, IBL-01313, IBL-03792 and/or IBL-00829, a Bt Cry1Ab (SEQ ID NO: 2) toxin, a Bt Cry1Ba (SEQ ID NO: 1) toxin or a chimeric Bt toxins as described herein, under conditions that permit expression of the insecticide nucleic acid.

### DESCRIPTION OF THE FIGURES

FIG. 1 is a graphical representation that shows the mortality induced by different toxins derived from the six Bt isolates, which showed ACP mortality when compared to controls. The six isolates were fed to ACP at a concentration of 500 μg protein/mL of diet. Data are presented as Mean±SEM (n=5). Significant differences with respect to diet control and buffer control treatments are indicated as *p<0.05 and **p<0.01 (One-way ANOVA, Tukey's test). Day 7 mortality ranged from 50 to 100% of the feeding ACP. When normalized to control mortality, the mortality induced by the toxins derived from each Bt strain at day 7 was: IBL-00829, 30% (the lowest); IBL-00200, 45%; IBL-00681, 50%; IBL-00048, 40%; IBL-00068, 70% (the highest); and IBL-00365, 60%.

FIG. 2 is a graphical representation which shows the estimated probability of mortality obtained by logistic regression of six strains tested at 500 μg protein/mL. The log odds of mortality were obtained using the lsmeans command within proc logistic. These were back transformed to the probability scale using the “ilink” option. Different letters indicate significant differences between groups (p<0.05). Toxins isolated from strains IBL-00200, -00681, -00048, -00068, and -00365 were all significantly different from the controls, but not from each other (group b). Strain IBL-00829 was not significantly different from either the controls or group b, representing an intermediate group (group ab).

FIG. 3 is a photographic representation which shows the profiles of trypsin-activated toxins derived from Bt isolates with toxicity to ACP: S: Soluble protein. A: Activated protein with 10% Trypsin for 1 h at 37° C. Proteins were separated by SDS-PAGE in a 12% gel and stained with Coomassie Blue R. Most of the pro-toxin proteins of the Bt isolates produced bands of approximately 25, 40, 75, or 120 kDa. Trypsin activated toxins ranged from 20 to 70 kDa, suggesting a diverse composition of Cry and possibly also Cyt toxins.

FIG. 4 is a graphical representation which shows the mortality induced by Cry toxins purified from IBL-00200: Cry1Ab (SEQ ID NO: 2) and Cry1Ba (SEQ ID NO: 1). Toxins were fed to ACP at 500 μg protein/mL of diet. Data are presented as Mean±SEM (n=5). Significant differences with respect to diet control and buffer control treatments are indicated as *p<0.05 and **p<0.01 (One-way ANOVA, Tukey's test). Both toxins caused significant mortality in ACP: Cry1Ba resulted in 60% mortality by day 4, Cry1Ab resulted in 60% mortality by day 6.

FIG. 5 is a graphical representation which shows the estimated probability of mortality obtained by logistic regression of the two individual toxins tested (Cry1Ab (SEQ ID NO: 2) and Cry1Ba (SEQ ID NO: 1)) at 500 μg protein/mL. The log odds of mortality were obtained using the lsmeans command within proc logistic. These were back transformed to the probability scale using the “ilink” option. Different letters indicate significant differences between groups (p<0.05). Cry1Ab (group ab) showed an intermediate level of mortality, being neither significantly different from either the control (group a) or Cry1Ba (group b). Cry1Ba, however, was significantly different from the control groups.

FIG. 6A-6B are graphical representations which shows the honeydew secretion score normalized to live animals at days 3, 7 and 11. Honeydew is produces as the ACP feed and was monitored as an indicator of feeding cessation. FIG. 6A shows the of exposure to Bt strains at 500 μg protein/mL. FIG. 6B shows the exposure of purified toxins at 500 μg protein/mL. Data for both FIG. 6A-6B are presented as Mean±SEM. The number of psyllids scored is indicated (n). Significant differences between the test group and the Buffer control are indicated by * (Pairwise non-parametric Dunn's test, p<0.05). ND: Not determined, due to 100% mortality. ACP fed on toxins derived from strain IBL-00068, -00200, and -00829 at a dose of 500 μg/mL produced significantly fewer excretions compared to the buffer control diet after 7 days of exposure. All insects in the IBL-00365 bioassay were dead by day 7, so data for this strain was not included in the analysis. This loss of honeydew indicates that there was a cessation of feeding induced by the Cry toxins. As for the individual, purified Cry1Ab (SEQ ID NO: 2) and Cry1Ba (SEQ ID NO: 1) toxins, a significate reduction in production of honeydew was noted for ACP fed on both after 11 days of exposure when compared to the Buffer control.

FIG. 7A-7C. are photographical representations which shows the identification of toxins in IBL-00200 by peptide sequencing. Peptide sequences obtained after trypsin treatment of bands A, B and C of strain IBL-00200 (FIG. 3) are highlighted. To activate the proteins, isolates were treated with 10% Trypsin for 1 h at 37° C. Proteins were separated by SDS-PAGE in a 12% gel and stained with Coomassie Blue R. The bands corresponding to A, B, and C were cut from the gel and then sequences using LC-MS/MS. Maximum likelihood analysis of the predicted protein sequences with those of holotype cry1 toxins predict that the tryptic fragments A (70 kDa), B (65 kDa), and C (60 kDa) are proteins highly similar to Cry1Bb (SEQ ID NO: 3), Cry1Ja (SEQ ID NO: 4), and Cry1Ab (SEQ ID NO: 2) respectively. FIG. 7A shows fragment A which is annotated as Cry1Bb (SEQ ID NO: 3). FIG. 7B shows fragment B which is annotated as Cry1Ja (SEQ ID NO: 4). FIG. 7C shows fragment C which is annotated as Cry1Ab (SEQ ID NO: 2).

FIG. 8 is a photographical representation which shows the pulldown assays of peptide-mCherry fusion proteins binding to ACP brush border membrane vesicle (BBMV). BBMV proteins were separated using a two-dimensional blot. After transferring the BBMV to PVDF Immobilon membranes and blocking, the membranes were incubated for 1 hr with the peptide-mCherry fusion protein. Detection for binding was performed using anti-mCherry antibody and the appropriate secondary. Pept-12 (SEQ ID NO: 5), pept-15 (SEQ ID NO: 6), pept-18 (SEQ ID NO: 7), and pept-22 (SEQ ID NO: 8) all showed specific binding to BBMV. A randomly selected peptide (Rand) and mCherry alone were used as negative controls.

FIG. 9 is a photographical representation which shows peptide 15 (SEQ ID NO: 6)-mCherry and peptide 18 (SEQ ID NO: 7)-mCherry bind in vivo to ACP gut. Dissected ACP guts were dissected and washed before excitation of mCherry and the resulting red fluorescence was visualized by a fluorescence microscope.

FIG. 10 is a diagrammatic and photographical representation which shows peptide 15-mCherry binds specifically to ACP BBMV using a competition assay. Unlabeled synthetic peptide in increasing amounts was used to compete with the fusion peptide for the BBMV. The anti-His antibody was used to detect only the His linker (SEQ ID NO: 30) on the fusion peptide.

FIG. 11A-11B are photographical representations which show the modeling sites for Cry1Ba (SEQ ID NO: 1) and Cry1Ab (SEQ ID NO: 2) engineering and where the addition of pept-15 (SEQ ID NO: 6) may be placed in external loops against ACP. FIG. 11A shows the Domain I, II, and III (DI, DII, and DIII respectively) locations in Cry1Ba of the sites for pept-15 additions. FIG. 11B shows the DI and DII locations sites in Cry1Ab for pept-15 additions.

FIG. 12 is a diagrammatic representation showing the identified ACP gut binding peptides and the structure of the peptide-mCherry fusion protein.

FIG. 13 is a diagrammatic and photographic representation showing the full blot of the peptide-mCherry fusion protein binding to BBMV for pept12 (SEQ ID NO: 5), pept15 (SEQ ID NO: 6), pept18 (SEQ ID NO: 7), and pept22 (SEQ ID NO: 8), as well as the specific binding of pept-12 through the peptide subunit.

### DETAILED DESCRIPTION OF THE INVENTION

The practice of the present invention will employ, unless otherwise indicated, conventional techniques of botany, microbiology, tissue culture, molecular biology, chemistry, biochemistry and recombinant DNA technology, which are within the skill of the art. Such techniques are explained fully in the literature. See, e.g., Langenheim and Thimann, (1982) Botany: Plant Biology and Its Relation to Human Affairs, John Wiley; Cell Culture and Somatic Cell Genetics of Plants, vol. 1, Vasil, ed. (1984); Stanier, et al., (1986) The Microbial World, 5^{th} ed., Prentice-Hall; Dhringra and Sinclair, (1985) Basic Plant Pathology Methods, CRC Press; Maniatis, et al., (1982) Molecular Cloning: A Laboratory Manual; DNA Cloning, vols. I and II, Glover, ed. (1985); Oligonucleotide Synthesis, Gait, ed. (1984); Nucleic Acid Hybridization, Hames and Higgins, eds. (1984); and the series Methods in Enzymology, Colowick and Kaplan, eds, Academic Press, Inc., San Diego, Calif.

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J Gen. Microbiol. 139:425-32) can be modified to yield a functionally identical molecule. Accordingly, each silent variation of a nucleic acid, which encodes a polypeptide of the present invention, is implicit in each described polypeptide sequence and incorporated herein by reference.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80% or 90%, preferably 60-90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton, Proteins, W.H. Freeman and Co. (1984).

As used herein, “consisting essentially of” means the inclusion of additional sequences to an object polynucleotide where the additional sequences do not selectively hybridize, under stringent hybridization conditions, to the same cDNA as the polynucleotide and where the hybridization conditions include a wash step in 0.1×SSC and 0.1% sodium dodecyl sulfate at 65° C.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolum (Yamao, et al., (1985) Proc. Natl. Acad. Sci. USA 82:2306-9), or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the rice preferred codon for a particular amino acid might be derived from known gene sequences from rice.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell, which comprises a heterologous nucleic acid sequence of the invention, which contains a vector and supports the replication and/or expression of the expression vector. Host cells may be prokaryotic cells such as E. coli, or eukaryotic cells such as yeast, insect, plant, amphibian, or mammalian cells. Preferably, host cells are monocotyledonous or dicotyledonous plant cells, including but not limited to maize, sorghum, sunflower, soybean, wheat, alfalfa, rice, cotton, canola, lawn grass, barley, millet, and tomato.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “nucleotide” is used herein as an adjective to describe molecules comprising RNA, DNA, or RNA/DNA hybrid sequences of any length in single-stranded or duplex form. More precisely, the expression “nucleotide sequence” encompasses the nucleic material itself and is thus not restricted to the sequence information (e.g. the succession of letters chosen among the four base letters) that biochemically characterizes a specific DNA or RNA molecule. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. The term “nucleotide” is also used herein to encompass “modified nucleotides” which comprise at least one modifications such as (a) an alternative linking group, (b) an analogous form of purine, (c) an analogous form of pyrimidine, or (d) an analogous sugar. For examples of analogous linking groups, purine, pyrimidines, and sugars see for example, WO 95/04064, which disclosure is hereby incorporated by reference in its entirety. Preferred modifications of the present invention include, but are not limited to, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylguanosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylguanosine, 5′-methoxycarboxymethyluracil, 5-methoxyuracil, 2-methylthio-N6-isopentenyladenine, uracil-5-oxyacetic acid (v) ybutoxosine, pseudouracil, guanosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N2-carboxypropyl)uracil, and 2,6-diaminopurine. The polynucleotide sequences herein may be prepared by any known method, including synthetic, recombinant, ex vivo generation, or a combination thereof, as well as utilizing any purification methods known in the art. Methylenemethylimino linked oligonucleotides as well as mixed backbone compounds, may be prepared as described in U.S. Pat. Nos. 5,378,825; 5,386,023; 5,489,677; 5,602,240; and 5,610,289. Formacetal and thioformacetal linked oligonucleotides may be prepared as described in U.S. Pat. Nos. 5,264,562 and 5,264,564. Ethylene oxide linked oligonucleotides may be prepared as described in U.S. Pat. No. 5,223,618. Phosphinate oligonucleotides may be prepared as described in U.S. Pat. No. 5,508,270. Alkyl phosphonate oligonucleotides may be prepared as described in U.S. Pat. No. 4,469,863. 3′-Deoxy-3′-methylene phosphonate oligonucleotides may be prepared as described in U.S. Pat. No. 5,610,289 or 5,625,050. Phosphoramidite oligonucleotides may be prepared as described in U.S. Pat. No. 5,256,775 or 5,366,878. Alkylphosphonothioate oligonucleotides may be prepared as described in WO 94/17093 and WO 94/02499. 3′-Deoxy-3′-amino phosphoramidate oligonucleotides may be prepared as described in U.S. Pat. No. 5,476,925. Phosphotriester oligonucleotides may be prepared as described in U.S. Pat. No. 5,023,243. Borano phosphate oligonucleotides may be prepared as described in U.S. Pat. Nos. 5,130,302 and 5,177,198.

By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, (1987) Guide To Molecular Cloning Techniques, from the series Methods in Enzymology, vol. 152, Academic Press, Inc., San Diego, Calif.; Sambrook, et al., (1989) Molecular Cloning: A Laboratory Manual, 2^{nd} ed., vols. 1-3; and Current Protocols in Molecular Biology, Ausubel, et al., eds, Current Protocols, a joint venture between Greene Publishing Associates, Inc. and John Wiley & Sons, Inc. (1994 Supplement).

As used herein “operably linked” includes reference to a functional linkage between a first sequence, such as a promoter, and a second sequence, wherein the promoter sequence initiates and mediates transcription of the DNA corresponding to the second sequence. Generally, operably linked means that the nucleic acid sequences being linked are contiguous and, where necessary to join two protein coding regions, contiguous and in the same reading frame.

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide,” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses, and bacteria which comprise genes expressed in plant cells such as Agrobacterium or Rhizobium. Examples are promoters that preferentially initiate transcription in certain tissues, such as leaves, roots, seeds, fibres, xylem vessels, tracheids, or sclerenchyma. Such promoters are referred to as “tissue-preferred.” A “cell type” specific promoter primarily drives expression in certain cell types in one or more organs, for example, vascular cells in roots or leaves. An “inducible” or “regulatable” promoter is a promoter which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated, and inducible promoters constitute the class of “non-constitutive” promoters. A “constitutive” promoter is a promoter which is active under most environmental conditions.

As used herein “recombinant” includes reference to a cell or vector that has been modified by the introduction of a heterologous nucleic acid, or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention; or may have reduced or eliminated expression of a native gene. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The terms “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass known analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity, and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's solution. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem., 138:267-84: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≥90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, part I, chapter 2, “Overview of principles of hybridization and the strategy of nucleic acid probe assays,” Elsevier, New York (1993); and Current Protocols in Molecular Biology, chapter 2, Ausubel, et al., eds, Greene Publishing and Wiley-Interscience, New York (1995). Unless otherwise stated, in the present application high stringency is defined as hybridization in 4×SSC, 5×Denhardt's solution (5 g Ficoll, 5 g polyvinypyrrolidone, 5 g bovine serum albumin in 500 mL of water), 0.1 mg/mL boiled salmon sperm DNA, and 25 mM Na phosphate at 65° C., and a wash in 0.1×SSC, 0.1% SDS at 65° C.

As used herein, “transgenic plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, “vector” includes reference to a nucleic acid used in transfection or transformation of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides or polypeptides: (a) “reference sequence,” (b) “comparison window,” (c) “sequence identity,” (d) “percentage of sequence identity,” and (e) “substantial identity.”

As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, and 50, 100 or longer. Those of ordinary skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Version 8 (available from Genetics Computer Group (GCG® programs (Accelrys, Inc., San Diego, Calif.).). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65, and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of ordinary skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As used interchangeably herein, the terms “nucleic acid molecule(s)”, “oligonucleotide(s)”, and “polynucleotide(s)” include RNA or DNA (either single or double stranded, coding, complementary or antisense), or RNA/DNA hybrid sequences of more than one nucleotide in either single chain or duplex form (although each of the above species may be particularly specified).

The terms “polypeptide” “peptide” and “protein”, used interchangeably herein, refer to a polymer of amino acids without regard to the length of the polymer; thus, peptides, oligopeptides, and proteins are included within the definition of polypeptide. This term also does not specify or exclude chemical or post-expression modifications of the polypeptides herein, although chemical or post-expression modifications of these polypeptides may be included excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination, and other modifications as known to one of ordinary skill the art. Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring.

Polypeptide sequence identity can be determined in the following manner. The subject polypeptide sequence is compared to a candidate polypeptide sequence using BLASTP (from the BLAST suite of programs, version 2.2.5 [November 2002]) in bl2seq, which is publicly available via the NCBI website. The default parameters of bl2seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available on the worldwide web, address ebi.ac.uk/emboss/align/, and GAP (Huang, X. (1994) On Global Sequence Alignment. Computer Applications in the Biosciences 10, 227-235.) as discussed above are also suitable global sequence alignment programs for calculating polypeptide sequence identity.

A preferred method for calculating polypeptide % sequence identity is based on aligning sequences to be compared using Clustal X (Jeanmougin et al., (1998) Trends Biochem. Sci. 23:403-5).

Polypeptide variants herein or used in the methods herein, also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.5 [November 2002]) from the NCBI website. The similarity of polypeptide sequences may be examined using the following unix command line parameters: bl2seq −i peptideseq1 −j peptideseq2 −F F −p blastp Variant polypeptide sequences preferably exhibit an E value of less than 1×10-6 more preferably less than 1×10-9, more preferably less than 1×10-12, more preferably less than 1×10-15, more preferably less than 1×10-18, more preferably less than 1×10-21, more preferably less than 1×10-30, more preferably less than 1×10-40, more preferably less than 1×10-50, more preferably less than 1×10-60, more preferably less than 1×10-70, more preferably less than 1×10-80, more preferably less than 1×10-90 and most preferably 1×10-100 when compared with any one of the specifically identified sequences.

The parameter −F F turns off filtering of low complexity sections. The parameter −p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., (1990) Science 247:1306).

As used herein, the terms “recombinant polynucleotide” and “polynucleotide construct” are used interchangeably to refer to linear or circular, purified or isolated polynucleotides that have been artificially designed and which comprise at least two nucleotide sequences that are not found as contiguous nucleotide sequences in their initial natural environment. In particular, these terms mean that the polynucleotide or cDNA is adjacent to “backbone” nucleic acid to which it is not adjacent in its natural environment. Additionally, to be “enriched” the cDNAs will represent 5% or more of the number of nucleic acid inserts in a population of nucleic acid backbone molecules. Backbone molecules according to the present invention include nucleic acids such as expression vectors, self-replicating nucleic acids, viruses, integrating nucleic acids, and other vectors or nucleic acids used to maintain or manipulate a nucleic acid insert of interest. Preferably, the enriched cDNAs represent 15% or more of the number of nucleic acid inserts in the population of recombinant backbone molecules. More preferably, the enriched cDNAs represent 50% or more of the number of nucleic acid inserts in the population of recombinant backbone molecules. In a highly preferred embodiment, the enriched cDNAs represent 90% or more (including any number between 90 and 100%, to the thousandth position, e.g., 99.5%) of the number of nucleic acid inserts in the population of recombinant backbone molecules.

As used herein, the term “operably linked” refers to a linkage of polynucleotide elements in a functional relationship. A sequence which is “operably linked” to a regulatory sequence such as a promoter means that said regulatory element is in the correct location and orientation in relation to the nucleic acid to control RNA polymerase initiation and expression of the nucleic acid of interest. For instance, a promoter or enhancer is operably linked to a coding sequence if it affects the transcription of the coding sequence.

A “promoter” refers to a DNA sequence recognized by the synthetic machinery of the cell, or introduced synthetic machinery, required to initiate the specific transcription of a gene. The phrase “under transcriptional control” can mean that the promoter is in the correct location and orientation in relation to the nucleic acid to control RNA polymerase initiation and expression of the gene in the isolated host cell of interest.

The term “chimeric” with reference to polypeptides encompasses recombinantly and synthetically produced polypeptides containing portions from different sources. Variant polypeptide sequences preferably exhibit at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a specifically exemplified sequence disclosed herein. Identity is found over a comparison window of at least 20 amino acid positions, preferably at least 50 amino acid positions, more preferably at least 100 amino acid positions, and most preferably over the entire length of an insecticidal polypeptide.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it is operably linked to promoter and terminator sequences.

A plant expressible promoter is one which directs transcription of an associated nucleotide sequence in a plant cell. It may be tissue-specific (e.g., phloem-specific or leaf or root specific) or it may be expressed in response to an environmental signal such as wounding or light. Alternatively, a plant expressible promoter may be constitutive, i.e., expressed in essentially all plant tissue.

A gut binding peptide (or multimer thereof) binds to the surface of the gut of an insect, especially the midgut. When incorporated into an insecticidal toxin (as part of an in-frame fusion) that does not normally bind and kill the insect, the gut binding peptide mediates the binding of the associated toxin to the gut and increases the toxicity of the protein to that insect. General references for cloning include Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989); Ausubel 1993, Current Protocols in Molecular Biology, Wiley, N.Y.

A modified insecticidal toxin is one in which there is a peptide portion which mediates binding of the chimeric toxin to an insect gut membrane and thus allows improved toxicity in a hemipteran insect.

A Bacillus thuringiensis-derived hemipteran insecticidal toxin or modified insecticidal toxin (protein) is one which kills at least one insect, in at least one stage of the development of that insect (larva, adult, for example). A specific example is the Bacillus thuringiensis Cry1Ba (SEQ ID NO: 1) or Cry1Ab (SEQ ID NO: 2), or toxin produced by BT IBL-00200, IBL-00068, IBL-00365, IBL-00681, IBL-00048, IBL-00937, IBL-01306, IBL-01313, IBL-03792 and/or IBL-00829, for which coding and amino acid sequences are provided herein and through sources such as Genbank and the like known to those of skill in the art.

The term “plant” is intended to include a whole plant, any part of a plant, a seed, a fruit, propagules and progeny of a plant.

As used herein, a “target insect” is an insect to be killed or inhibited in feeding by a chimeric insecticidal protein as described herein. Hemipteran and other sap-sucking insects are of particular importance in agriculture and horticulture, importantly Asian citrus psyllid (ACP).

Crop plants and agricultural plants are those of economic importance for human or animal food production or for animal fodder production and includes primarily citrus but can include grains, fruits and vegetables as well as grasses. Horticultural plants include those for turfgrass, windbreaks and landscaping and include ornamental plants such as flowers, shrubs, vines and the like.

### Gut Binding Proteins

The method described herein for identifying gut binding proteins from a phage library and demonstrating its activity are readily applied to isolating and characterizing other peptides that mediate binding to the guts of insects other than ACP, including, but not limited to, other insects within the order Hemiptera, members of which include aphids and planthoppers, white flies. In chimeric gut binding peptide-toxin proteins, toxicity is partly correlated with the extent of binding to the insect gut membrane.

A phage display library for peptides that bind to the gut of ACP, was screened, and gut binding peptides were isolated. Addition of one or more of these peptides and certain variants thereof to the ACP insecticidal model toxin, Cry1Ab (SEQ ID NO: 2), or Cry1Ba (SEQ ID NO: 1) is expected to result in significant insecticidal activity of the modified toxin, as compared with the naturally occurring toxin protein.

The addition of gut binding proteins such as peptide 12 (SEQ ID NO: 5), peptide 15 (SEQ ID NO: 6), peptide 18 (SEQ ID NO: 7), or peptide 22 (SEQ ID NO: 8) to Cry1Ab (SEQ ID NO: 2) or Cry1Ba (SEQ ID NO: 1) was highly effective in producing a hemipteran toxin, and addition of this sequence or a related gut binding peptide to a Cry toxin has the same effect. While the interaction of cytolytic toxins is lipid-based, Bt Cry toxins interact with specific insect gut receptors such as aminopeptidase N, cadherin and alkaline phosphatase (ALP), although the gut components bound by peptide 12 (SEQ ID NO: 5), peptide 15 (SEQ ID NO: 6), peptide 18 (SEQ ID NO: 7), and peptide 22 (SEQ ID NO: 8), have yet to be identified.

Also provided are plants which have been genetically engineered to contain within their genomes and express a chimeric insecticidal protein as described herein. Plants susceptible to attack by ACP of particular relevance include, without limitation, all citrus and closely-related species, as well as other hemipteran hosts such as plants susceptible to attack by aphids which include, without limitation, essentially all agricultural and food plants such as sugar beets, legumes, soybean (Glycine max), Chenopodium album, Chenopodium amaranticolor, Chenopodium quinoa, Cicer arietinum, Lathyrus odoratus, Lens culinaris, Lespedeza stipulacea, Lupinus albus, Lupinus angustifolius, Medicago Arabica, Medicago sativa, Melilotus albus, Nicotiana clevelandii, Phaseolus vulgaris, Pisum sativum, Trifolium hybridum, Trifolium incarnatum, Trifolium repens, Trifolium subterraneum, Vicia faba, Vicia sativa, and Vicia villosa, stone fruits, pears, peaches, grapes, berries, apples, wheat and other grains, tobacco, vegetables including, without limitation, tomato, potato, and essentially all horticultural and ornamental plants including roses (Rosa species), among others. Expression of a chimeric insecticidal protein or other chimeric insecticidal protein as described herein in these plants decreases plant damage due to hemipteran pests or other target insect. It is understood when the target insect is a sap-sucking insect, the chimeric insecticidal protein is expressed in the phloem fluids of that plant. For leaf-chewing insects and for certain insects, including hemipteran insects that feed on phloem or xylem, the chimeric insecticidal protein is expressed in the leaf and/or stems. Constitutive promoters which result in expression in essentially all plant tissues and promoters that are preferentially expressed in phloem, leaf, or root tissue or which are expressed in response to environmental signals such as light as well known to those of skill in the art.

Light-regulated promoters include, for example, those of the well-known genes encoding small subunit of ribulose-5-bisphosphate carboxylase of soybean, chlorophyll a binding protein, among others; see, e.g., U.S. Pat. Nos. 5,639,952; 5,656,496 and 5,750,385, among others.

For control of hemipteran insects that feed on phloem, phloem cell-specific or phloem-preferred promoters can be used to express a gut binding modified insecticidal toxin of interest in the phloem of transgenic plants. Phloem specific promoters known to the art include, without limitation, the CmGAS1 promoter described in U.S. Pat. No. 6,613,960; the Agrobacterium rhizogenes RoIC promoter (Graham et al., 1993); and the pumpkin PP2 promoter (Dinant et al. 2004).

Phloem-limited viruses have been described, including but not limited to the rice tungro virus (Bhattacharyya-Pakrasi et al., (1993) Plant J., 4:71-79) and the commelina yellow mottle virus (Medberry et al., (1992) Plant Cell, 4:185-192) also contain useful promoters that are active in vascular tissues. Others are described in U.S. Pat. No. 5,494,007; U.S. Pat. Nos. 5,824,857; 5,789,656; 6,613,960; and US Patent Publication 20100064394, and Guo et al., (2004) Transgenic Res, 13:559-566, Graham et al., (1997) Plant Molecular Biol, 33:729-735, among others.

Examples of additional useful phloem specific promoters include, but are not limited to, PP2-type gene promoters (U.S. Pat. No. 5,495,007), sucrose synthase promoters (Yang and Russell, (1990) Proc. Natl. Acad. Sci. USA 87:4144-4148,), glutamine synthetase promoters (Edwards et al., (1990) Proc. Natl. Acad. Sci. USA 87:3459-3463), and phloem-specific plasma membrane H+-ATPase promoters (DeWitt et al., (1991) Plant J. 1: 121-128), prunasin hydrolase promoters (U.S. Pat. No. 6,797,859), and a rice sucrose transporter (U.S. Pat. No. 7,186,821). For control of hemipteran pests that feed on xylem tissue, a variety of promoters that are active in xylem tissue including, but not limited to, protoxylem or metaxylem can be used.

One broad class of useful promoters is referred to as “constitutive” promoters in that they are active in most plant organs throughout plant development. For example, the promoter can be a viral promoter such as a CaMV35S or FMV35S promoter. Promoters that are active in certain plant tissues (i.e., tissue specific promoters) can also be used to drive expression of modified insecticidal toxin proteins as described herein. Since certain hemipteran insect pests are “piercing/sucking” insects that typically feed by inserting their proboscis into the vascular tissue of host plants, promoters that direct expression of insect inhibitory agents in the vascular tissue of the transgenic plants are particularly useful in the expression of a modified insecticidal toxin as described herein. Various Caulimovirus promoters, including but not limited to the CaMV35S, CaMV19S, FMV35S promoters and enhanced or duplicated versions thereof, typically deliver high levels of expression in vascular tissues and are thus useful for expression of a modified insecticidal toxin protein as described herein.

Promoters active in xylem tissue include, but are not limited to, promoters associated with phenylpropanoid biosynthetic pathways, such as the phenylalanine ammonia-lyase (PAL) promoters, cinnamate 4-hydroxylase (C4H) promoters, coumarate 3-hydroxylase promoters, O-methyl transferase (OMT) promoters, 4-coumarate:CoA ligase (4CL) promoters (U.S. Pat. No. 6,831,208), cinnamoyl-CoA reductase (CCR) promoters and cinnamyl alcohol dehydrogenase (CAD) promoters.

Further provided are methods for making a chimeric insecticidal protein, said method comprising the steps of isolating a peptide which binds to the gut of the target insect, inserting the gut-binding peptide into an insecticidal protein to product a chimeric insecticidal protein and verifying insecticidal activity in the target insect.

### Plant Transformation

Additional embodiments of the invention relate to transformed seeds and transgenic progeny plants of the parent transgenic plant, all expressing the gut-binding-peptide, peptide multimer or a fusion protein comprising same, advantageously expressed in the phloem of the plants, and the use of said plants, seeds, and plant parts in agro-industry and/or horticulture and/or in the production of food, feed, industrial products, oil, nutrients, and other valuable products. These other embodiments of the invention relate to transformed seed of such a plant, methods for breeding other plants using said plant, use of said plant in breeding or agriculture, and use of said plant to produce chemicals, food or feed products, as well as to reduce transmission of targeted virus diseases spread by sap-sucking insects and to reduce plant damage and economic losses due to those targeted virus diseases. The use of transgenic plants expressing the peptide, peptide multimer or fusion protein among, near or surrounding a nontransgenic plant of interest also affords some protection to the nontransgenic plant of interest.

### Insecticidal Peptides

The present disclosure relates, according to some embodiments, to peptides and/or proteins having insecticidal activity. In some embodiments, the toxin or chimeric toxin may be small (about 5 kDa), may be basic and/or may be cysteine-rich. In some embodiments, the toxin may comprise a peptide having an amino acid sequence sharing at least about 95% identity, at least about 96% identity, at least about 97% identity, at least about 98% identity, at least about 99% identity, and/or about 100% identity with the sequences disclosed herein. In some embodiments, an insecticidal peptide may further comprise one or more amino acids that are independently and/or collectively either neutral (e.g., do not adversely impact antibacterial functionality) and/or augment antibacterial functionality (e.g., by directing the peptide to a desired location (e.g., cellular and/or extracellular).

### Nucleic Acids

The present disclosure relates, in some embodiments, to nucleic acids (e.g., cassettes, vectors) comprising one or more sequences encoding one or more insecticidal peptides. For example, a nucleic acid may comprise a cassette comprising a synthetic nucleic acid sequence of Cry1Ab (SEQ ID NO: 2), Cry1Ba (SEQ ID NO: 1) or chimeric proteins with gut binding with codons optimized or citrus codon usage. A nucleic acid may comprise a sequence encoding a signal peptide (e.g., PR-1b). In some embodiments, expression of a nucleic acid comprising a sequence encoding an insecticidal peptide may be optimized by positioning an initiation codon in a favorable (e.g., optimal) 5′ context. According to some embodiments, a nucleic acid may comprise an expression control sequence (e.g., operably linked to a coding sequence). For example, a nucleic acid may comprise a coding gene sequence under the control of a dual enhanced CaMV 35S promoter with a 5′ UTR from TEV plant potyvirus (e.g., to provide a translation-enhancing activity to the defensin genes).

According to some embodiments, a nucleic acid may comprise a nucleotide sequence having at least about 75% at least about 80% at least about 85% at least about 90%, at least about 95% at least about 97% identity, at least about 98% at least about 99%, and/or about 100% identity to sequences disclosed herein. A nucleotide sequence may encode, in some embodiments, an amino acid sequence having at least about 98% at least about 99% and/or about 100% identity to sequences disclosed herein.

A nucleic acid sequence, according to some embodiments, may hybridize to a nucleic acid having the nucleotide sequence disclosed herein under stringent conditions. Stringent conditions may include, for example, (a) 4×SSC at 65° C. followed by 0.1×SSC at 65° for 60 minutes and/or (b) 50% formamide, 4×SSC at 65° C. A nucleic acid may comprise a deletion fragment (e.g., a deletion of from about 1 to about 12 bases) of a nucleic acid having a sequence disclosed herein that retains insecticidal activity against at least one hemipteran organism capable of infecting a citrus plant. One of ordinary skill in the art having the benefit of the present disclosure may prepare one or more deletion fragments of a nucleic acid having a sequence disclosed herein and screen the resulting fragments for insecticidal activity against at least one microorganism capable of infecting a citrus plant.

A nucleic acid sequence having a sequence like those disclosed herein may be identified by database searches using the sequence or elements thereof as the query sequence using the Gapped BLAST algorithm (Altschul et al., (1997) Nucl. Acids Res. 25:3389-3402) with the BLOSUM62 Matrix, a gap cost of 11 and persistence cost of 1 per residue and an E value of 10. Sequence identity may be assessed by any available method according to some embodiments. For example, two sequences may be compared with either ALIGN (Global alignment) or LALIGN (Local homology alignment) in the FASTA suite of applications (Pearson and Lipman, (1988) Proc. Nat. Acad. Sci., 85:2444-2448; Pearson, (1990) Methods in Enzymology, 183:63-98) with the BLOSUM50 matrix and gap penalties of −16, −4. Sequence similarity may be assessed according to ClustalW (Larkin et al., (2007) Bioinformatics, 23(21): 2947-2948), BLAST, FASTA or similar algorithm.

### Expression Cassettes and Vectors

The disclosure relates, in some embodiments, to expression vectors and/or expression cassettes for expressing a nucleic acid sequence (e.g., a coding sequence) in a cell and comprising an expression control sequence and the nucleic acid sequence operably linked to the expression control sequence. Thus, for example, an expression cassette may comprise a heterologous coding sequence, the expression of which may be desired in a plant.

### Expression Vectors

The disclosure relates, in some embodiments, to an expression vector which may comprise, for example, a nucleic acid having an expression control sequence and a coding sequence operably linked to the expression control sequence. In some embodiments, an expression control sequence may comprise one or more promoters, one or more operators, one or more enhancers, one or more ribosome binding sites, and/or combinations thereof. An expression control sequence may comprise, for example, a nucleic acid having promoter activity. An expression control sequence, according to some embodiments, may be constitutively active or conditionally active in (a) an organ selected from root, leaf, stem, flower, seed, and/or fruit, and/or (b) active in a tissue selected from epidermis, periderm, parenchyma, collenchyma, sclerenchyma, xylem, phloem, and/or secretory structures. An expression control sequence, according to some embodiments, may be operable to drive expression of a nucleic acid sequence (e.g., a coding sequence) in a cell. Metrics for expression may include, for example, rate of appearance and/or accumulation of a gene product (e.g., RNA and/or protein) and/or total accumulation of a gene product as of one or more time points (e.g., elapsed time after a starting point and/or a stage of development). Comparative assays for gene products may be qualitative, semi-quantitative, and/or quantitative in some embodiments. Comparative assays may indirectly and/or directly assess the presence and/or amount of gene product. In some embodiments, an expression control sequence may be sensitive to one or more stimuli (e.g., one or more small molecules, one or more plant defense-inducing agents, mechanical damage, temperature, pressure). For example, activity of an expression control sequence may be enhanced or suppressed upon infection with a microorganism (e.g., a bacteria or a virus).

An expression vector may be contacted with a cell (e.g., a plant cell) under conditions that permit expression (e.g., transcription) of the coding sequence. Examples of expression vectors may include the Agrobacterium transformation constructs. An expression control sequence may be contacted with a plant cell (e.g., an embryonic cell, a stem cell, a callous cell) under conditions that permit expression of the coding sequence in the cell and/or cells derived from the plant cell according to some embodiments. An expression vector may be contacted with a cell (e.g., a plant cell), in some embodiments, under conditions that permit inheritance of at least a portion of the expression vector in the cell's progeny. According to some embodiments, an expression vector may include one or more selectable markers. For example, an expression vector may include a marker for selection when the vector is in a bacterial host, a yeast host, and/or a plant host.

### Expression Cassettes

According to some embodiments, the disclosure relates to an expression cassette which may comprise, for example, a nucleic acid having an expression control sequence and a coding sequence operably linked to the expression control sequence. An expression cassette may be comprised in an expression vector. A coding sequence, in some embodiments, may comprise any coding sequence expressible in at least one plant cell. For example, a coding sequence may comprise a plant sequence, a yeast sequence, a bacterial sequence, a viral sequence (e.g., plant virus), an artificial sequence, an antisense sequence thereof, a fragment thereof, a variant thereof, and/or combinations thereof. A coding sequence may comprise, in some embodiments, a sequence encoding one or more gene products with insecticidal, activity. A coding sequence may comprise, in some embodiments, a start codon, an intron, and/or a translation termination sequence. According to some embodiments, a coding sequence may comprise one or more natural or artificial coding sequences (e.g., encoding a single protein or a chimera). According to some embodiments, an expression cassette may optionally comprise a termination sequence. A coding sequence, in some embodiments, may comprise a sequence at least partially codon optimized for expression in an organism of interest (e.g., a citrus plant).

An expression control sequence may be used, in some embodiments, to construct an expression cassette comprising, in the 5′ to 3′ direction, (a) the expression control sequence, (b) a heterologous gene or a coding sequence, or sequence complementary to a native plant gene under control of the expression control sequence, and/or (c) a 3′ termination sequence (e.g., a termination sequence comprising a polyadenylation site). An expression cassette may be incorporated into a variety of autonomously replicating vectors in order to construct an expression vector. An expression cassette may be constructed, for example, by ligating an expression control sequence to a sequence to be expressed (e.g., a coding sequence).

Some techniques for construction of expression cassettes are well known to those of ordinary skill in the art. For example, a variety of strategies are available for ligating fragments of DNA, the choice of which depends on the nature of the termini of the DNA fragments. An artisan of ordinary skill having the benefit of the present disclosure, a coding sequence (e.g., having insecticidal activity) and/or portions thereof may be provided by other means, for example chemical or enzymatic synthesis. A nucleic acid may comprise, in a 5′ to 3′ direction, an expression control sequence, a linker (optional), and a coding sequence according to some embodiments. A nucleic acid may comprise, in some embodiments, one or more restriction sites and/or junction sites between an expression control sequence, a linker, and/or a coding sequence.

### Plants

The present disclosure relates, in some embodiments, to a plant cell (e.g., an embryonic cell, a stem cell, a callous cell), a tissue, and/or a plant comprising an insecticidal peptide (e.g., a heterologous insecticidal peptide), and/or a nucleic acid (e.g., a heterologous and/or expressible nucleic acid) comprising a nucleic acid sequence encoding an insecticidal peptide. A plant and/or plant cell may be a dicot in some embodiments. Examples of a dicot may include, without limitation, coffee, tomato, pepper, tobacco, lima bean, Arabidopsis, rubber, orange, grapefruit, lemon, lime, tangerine, mandarin, pummelo, potato, squash, peas, and/or sugar beet. A plant cell may be included in a plant tissue, a plant organ, and/or a whole plant in some embodiments. A plant cell in a tissue, organ, and/or whole plant may be adjacent, according to some embodiments, to one or more isogenic cells and/or one or more heterogenic cells. In some embodiments, a plant may include primary transformants and/or progeny thereof. A plant comprising a nucleic acid (e.g., a heterologous and/or expressible nucleic acid) comprising a nucleic acid sequence encoding an insecticidal peptide may further comprise an expression control sequence operably linked to the nucleic acid, in some embodiments. A nucleic acid sequence encoding an insecticidal peptide may be expressed, according to some embodiments, in a plant in one or more up to all (e.g., substantially all) organs, tissues, and/or cell types including, without limitation, stalks, leaves, roots, seeds, flowers, fruit, meristem, parenchyma, storage parenchyma, collenchyma, sclerenchyma, epidermis, mesophyll, bundle sheath, guard cells, protoxylem, metaxylem, phloem, phloem companion, and/or combinations thereof. In some embodiments, a nucleic acid and/or its gene product (e.g., an insecticidal peptide) may be located in and/or translocated to one or more organelles (e.g., vacuoles, chloroplasts, mitochondria, plastids).

### Transforming a Plant

The present disclosure relates, according to some embodiments, to methods for independent transformation of citrus (e.g., a native genome of a citrus plant). For example, a method may comprise independent transformation, using Agrobacterium tumefaciens (At), of the native genome of the orange (Citrus sinensis) cultivars “Rohde Red”, “Hamlin”, and/or “Marrs.” A transformation method may comprise contacting a nucleic acid comprising insecticidal sequence with a citrus plant according to some embodiments. A transformed plant (e.g., a transformed genome of a new orange cultivar) may independently contain, in some embodiments a sequence of an insecticidal gene encoding a toxin not found within the native gene pool of the Citrus genus.

### Grafting

The present disclosure relates to grafting at least a portion of a first plant (e.g., a citrus plant) with at least a portion of a second plant (e.g., a citrus plant), according to some embodiments. A first plant may be in any desired condition including, without limitation, a healthy condition, a diseased condition, an injured condition, a stressed condition (e.g., heat, cold, water, and the like), and/or combinations thereof. A first plant may have any desired genotype including, without limitation, wild type, transgenic, mutant, and/or the like with respect to a gene and/or trait of interest.

A second plant may be in any desired condition including, without limitation, a healthy condition, a diseased condition, an injured condition, a stressed condition (e.g., heat, cold, water, and the like), and/or combinations thereof. A second plant may have any desired genotype including, without limitation, wild type, transgenic, mutant, and/or the like with respect to a gene and/or trait of interest. A first and/or a second plant may comprise at least one insecticidal peptide and/or at least one nucleic acid comprising a sequence encoding at least one insecticidal peptide. Where both a first plant comprises at least one insecticidal peptide and/or at least one nucleic acid comprising a sequence encoding at least one insecticidal peptide and a second plant comprises at least one insecticidal peptide and/or at least one nucleic acid comprising a sequence encoding at least one insecticidal peptide, it may be desirable for the first and second plants to have the same and/or different insecticidal peptides and/or nucleic acids encoding insecticidal peptides. Grafting may comprise cutting a portion of a first plant to form a fresh cut site, cutting a portion of a second plant to create a second cut site, and/or contacting a first cut site with a second cut site. A cut site may comprise at least one vascular bundle. Grafting may comprise forming a graft junction and/or, optionally, sealing the graft junction (e.g., by coating the periphery of the graft junction with one or more barrier materials).

### Preventing or Ameliorating Plant Disease

The present disclosure relates, in some embodiments, to compositions, organisms, systems, and methods for preventing, ameliorating, and/or treating a plant disease (e.g., a citrus disease) and/or at least one symptom of a plant disease. For example, a method may comprise grafting at least a portion of a plant (e.g., a citrus plant) having a plant disease and/or expressing at least one symptom of a plant disease with at least a portion of a plant (e.g., a citrus plant) comprising an insecticidal peptide. Examples of a plant disease include, without limitation, citrus huanglongbing (ex. greening) caused by Candidatus Liberibacter asiaticus (Las). According to some embodiments, preventing, ameliorating, and/or treating a plant disease (e.g., a citrus disease) and/or at least one symptom of a plant disease may comprise treating and/or curing one or more devastating bacterial diseases of citrus. For example, plants comprising stably integrated Bt toxins with gut binding protein transgenes in expressible form may help to reduce or eliminate infection by insects such as Hemiptera which carry disease causing pathogens

According to some embodiments, the present disclosure relates to compositions, organisms, systems, and methods for augmenting a plant's native resistance to and/or conferring on a plant resistance to insect infestation and resultant disease (e.g., a citrus disease). For example, a method may comprise contacting a plant with an insecticidal peptide and/or an expressible nucleic acid comprising a nucleic acid sequence encoding an insecticidal peptide. An expressible nucleic acid comprising a nucleic acid sequence encoding an insecticidal peptide may be and/or comprise an expression cassette in some embodiments. Contacting may comprise, according to some embodiments, grafting at least a portion of a target plant with a plant comprising an insecticidal peptide and/or an expressible nucleic acid comprising a nucleic acid sequence encoding an insecticidal peptide. In some embodiments, contacting may comprise contacting at least a portion of a target plant with a vector (e.g., via Agrobacterium-mediated transformation) comprising an insecticidal peptide and/or an expressible nucleic acid comprising a nucleic acid sequence encoding an insecticidal peptide. Examples of a plant disease include, without limitation, huanglongbing (ex. greening) caused by Candidatus Liberibacter asiaticus (Las).

### Making a Citrus-Expressible Insecticidal Peptide

In some embodiments, the present disclosure relates to compositions, organisms, systems, and methods for forming a citrus-expressible nucleic acid comprising a nucleic acid sequence encoding at least one Bt Cry1Ab (SEQ ID NO: 2), Cry1Ba (SEQ ID NO: 1) or chimeric Bt insecticidal peptide. For example, a method may comprise identifying an amino acid sequence of an insecticidal peptide of interest, reverse translating the amino acid sequence to produce a first nucleic acid sequence; codon-optimizing the first nucleic acid sequence for expression in citrus to produce a second nucleic acid sequence, and/or synthesizing a nucleic acid having the second nucleic acid sequence. A method may comprise, in some embodiments, covalently bonding a nucleic acid having the second nucleic acid sequence with one or more nucleic acids having expression control sequences that are operable in citrus in an operable orientation and/or position relative to the nucleic acid having the second nucleic acid sequence.

### Construction of Nucleic Acids

The isolated nucleic acids of the present invention can be made using (a) standard recombinant methods, (b) synthetic techniques, or combinations thereof. In some embodiments, the polynucleotides of the present invention will be cloned, amplified, or otherwise constructed from a fungus or bacteria.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. The nucleic acid of the present invention—excluding the polynucleotide sequence—is optionally a vector, adapter, or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present invention less the length of its polynucleotide of the present invention is less than 20 kilobase pairs, often less than 15 kb, and frequently less than 10 kb. Use of cloning vectors, expression vectors, adapters, and linkers is well known in the art. Exemplary nucleic acids include such vectors as: M13, lambda ZAP Express, lambda ZAP II, lambda gt10, lambda gt11, pBK-CMV, pBK-RSV, pBluescript II, lambda DASH II, lambda EMBL 3, lambda EMBL 4, pWE15, SuperCos 1, SurfZap, Uni-ZAP, pBC, pBS+/−, pSG5, pBK, pCR-Script, pET, pSPUTK, p3′SS, pGEM, pSK+/−, pGEX, pSPORTI and II, pOPRSVI CAT, pOPI3 CAT, pXT1, pSG5, pPbac, pMbac, pMC1neo, pOG44, pOG45, pFRTβGAL, pNEOβGAL, pRS403, pRS404, pRS405, pRS406, pRS413, pRS414, pRS415, pRS416, lambda MOSSlox, and lambda MOSElox. Optional vectors for the present invention, include but are not limited to, lambda ZAP II, and pGEX. For a description of various nucleic acids see, e.g., Stratagene Cloning Systems, Catalogs 1995, 1996, 1997 (La Jolla, Calif.); and, Amersham Life Sciences, Inc, Catalog '97 (Arlington Heights, Ill.).

### Synthetic Methods for Constructing Nucleic Acids

The isolated nucleic acids of the present invention can also be prepared by direct chemical synthesis by methods such as the phosphotriester method of Narang, et al., (1979) Meth. Enzymol. 68:90-9; the phosphodiester method of Brown, et al., (1979) Meth. Enzymol. 68:109-51; the diethylphosphoramidite method of Beaucage, et al., (1981) Tetra. Letts. 22(20):1859-62; the solid phase phosphoramidite triester method described by Beaucage, et al., supra, e.g., using an automated synthesizer, e.g., as described in Needham-VanDevanter, et al., (1984) Nucleic Acids Res. 12:6159-68; and, the solid support method of U.S. Pat. No. 4,458,066. Chemical synthesis generally produces a single stranded oligonucleotide. This may be converted into double stranded DNA by hybridization with a complementary sequence or by polymerization with a DNA polymerase using the single strand as a template. One of skill will recognize that while chemical synthesis of DNA is limited to sequences of about 100 bases, longer sequences may be obtained by the ligation of shorter sequences.

PCR is used to confirm that the transgenic plants contain the proper insertion of the chimeric toxin gene. RT-PCR is used to confirm that the peptide, tandem repeat or fusion protein mRNA is transcribed, and ELISA or western blots establish that the protein is expressed.

The effectiveness of the expressed modified insecticidal toxin is shown by bioassay of the transformed plants, as described herein.

### Genome Editing Using Site-Specific Nucleases

Genome editing may also be used to create plants expressing the insecticidal toxins herein. Genome editing uses engineered nucleases such as RNA guided DNA endonucleases or nucleases composed of sequence specific DNA binding domains fused to a non-specific DNA cleavage module. These engineered nucleases enable efficient and precise genetic modifications by inducing targeted DNA double stranded breaks that stimulate the cell's endogenous cellular DNA repair mechanisms to repair the induced break. Such mechanisms include, for example, error prone non-homologous end joining (NHEJ) and homology directed repair (HDR).

In the presence of donor plasmid with extended homology arms, HDR can lead to the introduction of single or multiple transgenes.

Engineered nucleases useful in the methods include zinc finger nucleases (ZFNs), transcription activator-like (TAL) effector nucleases (TALEN) and CRISPR/Cas9 type nucleases.

Typically, nuclease encoded genes are delivered into cells by plasmid DNA, viral vectors or in vitro transcribed mRNA.

### Insecticidal Compositions

Compositions of the invention will include the insecticidal proteins identified herein in an effective amount to kill or otherwise inhibit Hemiptera. The effective amount of the composition would comprise a peptide with 90% amino acid sequence identity or greater with SEQ ID NO: 1 or SEQ ID NO: 2. It is contemplated that such a composition is applied to the food source in the environment of the hemipteran pests. The composition may be formulated for preventive or prophylactic application to an area to prevent infestation of pests.

In another embodiment, a method for inhibiting hemipteran insect pests is included herewith, the method comprising selecting a Bacillus thuringiensis Cry1Ba (SEQ ID NO: 1), Cry1Ba and Cry1Ab (SEQ ID NO: 2) or toxin produced by Bt isolate IBL-00200, IBL-00068, IBL-00365, IBL-00681, IBL-00048, IBL-00937, IBL-01306, IBL-01313, IBL-03792 and/or IBL-00829, and applying an effective amount of said protein to the insect pest, wherein the mortality of said insect increases.

In a particularly preferred embodiment the Hemiptera-active toxins are modified to create chimeric proteins designed for uptake by Hemiptera and other sap-sucking insects. There is provided a modified insecticidal toxin that specifically binds to a receptor in a sap-sucking insect gut, especially an Asian citrus psyllid, via a peptide or peptide multimers incorporated within the modified insecticidal toxin, for example as an N-terminal extension of the toxin or incorporated within surface domains of the toxin. By mediating the gut binding of the insecticidal protein, the toxin is acquired by the insect feeding on a plant which expresses the chimeric toxin or to which the chimeric toxin has been topically applied, certain plant pests feeding on the plant are killed, although topical application of a modified insecticidal toxin is not considered appropriate for sap sucking-insects. The use of this chimeric toxin disclosed herein applies primarily to insects which feed on plant fluids (sap-sucking insects), including but not limited to, aphids and planthoppers, whiteflies (Hemiptera) and most particularly the Asian citrus psyllid, Diaphorina citri.

### Further Additives

One aspect of the present invention is to provide a composition as described above that can be used as a feeding source and injected into plants, or added to create an alternate feeding source, additionally comprising at least one auxiliary selected from the group consisting of extenders, solvents, spontaneity promoters, carriers, emulsifiers, dispersants, frost protectants, thickeners and adjuvants. Those compositions are referred to as formulations and may be added to a food source for the insects.

Accordingly, in one aspect of the present invention such formulations, and application forms prepared from them, are provided as crop protection agents and/or pesticidal agents, such as drench, drip and spray liquors, comprising the composition of the invention. The application forms may comprise further crop protection agents and/or pesticidal agents, and/or activity-enhancing adjuvants such as penetrants, examples being vegetable oils such as, for example, rapeseed oil, sunflower oil, mineral oils such as, for example, liquid paraffins, alkyl esters of vegetable fatty acids, such as rapeseed oil or soybean oil methyl esters, or alkanol alkoxylates, and/or spreaders such as, for example, alkylsiloxanes and/or salts, examples being organic or inorganic ammonium or phosphonium salts, examples being ammonium sulphate or diammonium hydrogen phosphate, and/or retention promoters such as dioctyl sulphosuccinate or hydroxypropylguar polymers and/or humectants such as glycerol and/or fertilizers such as ammonium, potassium or phosphorous fertilizers, for example.

Examples of typical formulations include water-soluble liquids (SL), emulsifiable concentrates (EC), emulsions in water (EW), suspension concentrates (SC, SE, FS, OD), water-dispersible granules (WG), granules (GR) and capsule concentrates (CS); these and other possible types of formulation are described, for example, by Crop Life International and in Pesticide Specifications, Manual on development and use of FAO and WHO specifications for pesticides, FAO Plant Production and Protection Papers-173, prepared by the FAO/WHO Joint Meeting on Pesticide Specifications, 2004, ISBN: 9251048576. The formulations may comprise active agrochemical compounds other than one or more active compounds of the invention.

The formulations or application forms in question preferably comprise auxiliaries, such as extenders, solvents, spontaneity promoters, carriers, emulsifiers, dispersants, frost protectants, biocides, thickeners and/or other auxiliaries, such as adjuvants, for example. An adjuvant in this context is a component which enhances the biological effect of the formulation, without the component itself having a biological effect. Examples of adjuvants are agents which promote the retention, spreading, attachment to the leaf surface, or penetration.

These formulations are produced in a known manner, for example by mixing the active compounds with auxiliaries such as, for example, extenders, solvents and/or solid carriers and/or further auxiliaries, such as, for example, surfactants. The formulations are prepared either in suitable plants or else before or during the application.

Suitable for use as auxiliaries are substances which are suitable for imparting to the formulation of the active compound or the application forms prepared from these formulations (such as, e.g., usable crop protection agents, such as spray liquors or seed dressings) particular properties such as certain physical, technical and/or biological properties.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

In principle it is possible to use all suitable solvents. Suitable solvents are, for example, aromatic hydrocarbons, such as xylene, toluene or alkylnaphthalenes, for example, chlorinated aromatic or aliphatic hydrocarbons, such as chlorobenzene, chloroethylene or methylene chloride, for example, aliphatic hydrocarbons, such as cyclohexane, for example, paraffins, petroleum fractions, mineral and vegetable oils, alcohols, such as methanol, ethanol, isopropanol, butanol or glycol, for example, and also their ethers and esters, ketones such as acetone, methyl ethyl ketone, methyl isobutyl ketone or cyclohexanone, for example, strongly polar solvents, such as dimethyl sulphoxide, and water.

All suitable carriers may in principle be used. Suitable carriers are in particular: for example, ammonium salts and ground natural minerals such as kaolins, clays, talc, chalk, quartz, attapulgite, montmorillonite or diatomaceous earth, and ground synthetic minerals, such as finely divided silica, alumina and natural or synthetic silicates, resins, waxes and/or solid fertilizers. Mixtures of such carriers may likewise be used. Carriers suitable for granules include the following: for example, crushed and fractionated natural minerals such as calcite, marble, pumice, sepiolite, dolomite, and also synthetic granules of inorganic and organic meals, and also granules of organic material such as sawdust, paper, coconut shells, maize cobs and tobacco stalks.

Liquefied gaseous extenders or solvents may also be used. Particularly suitable are those extenders or carriers which at standard temperature and under standard pressure are gaseous, examples being aerosol propellants, such as halogenated hydrocarbons, and also butane, propane, nitrogen and carbon dioxide.

Examples of emulsifiers and/or foam-formers, dispersants or wetting agents having ionic or nonionic properties, or mixtures of these surface-active substances, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyltaurates), phosphoric esters of polyethoxylated alcohols or phenols, fatty acid esters of polyols, and derivatives of the compounds containing sulphates, sulphonates and phosphates, examples being alkylaryl polyglycol ethers, alkylsulphonates, alkyl sulphates, arylsulphonates, protein hydrolysates, lignin-sulphite waste liquors and methylcellulose. The presence of a surface-active substance is advantageous if one of the active compounds and/or one of the inert carriers is not soluble in water and if application takes place in water.

Further auxiliaries that may be present in the formulations and in the application forms derived from them include colorants such as inorganic pigments, examples being iron oxide, titanium oxide, Prussian Blue, and organic dyes, such as alizarin dyes, azo dyes and metal phthalocyanine dyes, and nutrients and trace nutrients, such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present. Additionally, present may be foam-formers or defoamers.

Furthermore, the formulations and application forms derived from them may also comprise, as additional auxiliaries, stickers such as carboxymethylcellulose, natural and synthetic polymers in powder, granule or latex form, such as gum arabic, polyvinyl alcohol, polyvinyl acetate, and also natural phospholipids, such as cephalins and lecithins, and synthetic phospholipids. Further possible auxiliaries include mineral and vegetable oils.

There may possibly be further auxiliaries present in the formulations and the application forms derived from them. Examples of such additives include fragrances, protective colloids, binders, adhesives, thickeners, thixotropic substances, penetrants, retention promoters, stabilizers, sequestrants, complexing agents, humectants and spreaders. Generally speaking, the active compounds may be combined with any solid or liquid additive commonly used for formulation purposes.

Suitable retention promoters include all those substances which reduce the dynamic surface tension, such as dioctyl sulphosuccinate, or increase the viscoelasticity, such as hydroxypropylguar polymers, for example.

Suitable penetrants in the present context include all those substances which are typically used in order to enhance the penetration of active agrochemical compounds into plants. Penetrants in this context are defined in that, from the (generally aqueous) application liquor and/or from the spray coating, they are able to penetrate the cuticle of the plant and thereby increase the mobility of the active compounds in the cuticle. This property can be determined using the method described in the literature (Baur et al., (1997) Pesticide Science, 51:131-152). Examples include alcohol alkoxylates such as coconut fatty ethoxylate (10) or isotridecyl ethoxylate (12), fatty acid esters such as rapeseed or soybean oil methyl esters, fatty amine alkoxylates such as tallowamine ethoxylate (IS), or ammonium and/or phosphonium salts such as ammonium sulphate or diammonium hydrogen phosphate, for example.

The formulations preferably comprise between 0.00000001% and 98% by weight of active compound or, with particular preference, between 0.01% and 95% by weight of active compound, more preferably between 0.5% and 90% by weight of active compound, based on the weight of the formulation. The content of the active compound is defined as the sum of the at least one biological control agent and the at least one insecticide.

The active compound content of the application forms (crop protection products) prepared from the formulations may vary within wide ranges. The active compound concentration of the application forms may be situated typically between 0.00000001% and 95% by weight of active compound, preferably between 0.00001% and 1% by weight, based on the weight of the application form.

All references and patent documents cited herein reflect the level of skill in the relevant arts and are incorporated by reference in their entireties to the extent there is no inconsistency with the present disclosure. The examples provided herein are for illustrative purposes and are not intended to limit the scope of the invention as claimed. Any variations in the exemplified compositions, plants and methods which occur to the skilled artisan are intended to fall within the scope of the present invention.

### Example 1

Identification of Bacillus thuringiensis Strains and Individual Toxins with Activity Against Asian Citrus Psyllid, Diaphorina citri (Hemiptera)

In this example, we screened multiple strains of Bacillus thuringiensis (Bt) for toxicity against Asian citrus psyllid (ACP) to provide additional control methods for the ACP vector that causes huanglonging (HLB). Bt produces multiple toxins with activity against a diverse range of insects. Bt δ-endotoxins, which include the crystal (Cry) proteins damage the midgut following ingestion and have been used successfully for management of other insect pests (Shelton et al. 2002; Christou et al. 2006). The Cry proteins are pore-forming toxins, although the mechanism of action is poorly understood (Vachon et al., (2012) J Invertebr Pathol, 111:1-12; Palma et al., (2014) Toxins, 6:3296-3325). While individual Cry toxins are generally toxic to a particular order of insects, collectively they exhibit activity across orders, particularly the Lepidoptera, Diptera and Coleoptera. A major limitation for use of Bt toxins against ACP is the lack of information on the efficacy of Bt toxins against ACP. Some Bt toxins have activity against sap sucking insects with, for example, low-level toxicity against aphids (Hemiptera) (Chougule and Bonning, (2012) Toxins, 4:405-429), and high toxicity against plant bugs, Lygus hesperus (Baum et al., (2010) U.S. Pat. No. 8,609,936B2; Baum et al., (2012) J Econ Entomol, 105:616-624). An ACP-active toxin could be delivered to the ACP feeding site (primarily the phloem) via use of transgenic citrus, root drenches, trunk injections, or use of a non-pathogenic phloem-limited virus such as the Citrus tristeza virus vector (Hajeri et al., (2014) J Biotechnol, 176:42-49).

The overall goal of this study was to identify a Bt crystal toxin with toxicity to ACP. Toxins derived from Bt could be used for effective management of the psyllid and associated HLB disease to the benefit of both the citrus industry and the environment as a more sustainable management approach than the use of non-specific chemical insecticides.

### Bt Strains and Toxins

We selected six strains of Bt that were toxic to ACP and two recombinant Cry toxins. To test for toxicity Bt strains used in this study were selected from the collection of the Invasive Insect Biocontrol and Behavior Laboratory (USDA-ARS, Beltsville, Md.). Bt spores stored on paper disks were germinated by placing the disks on L-agar at 30° C. At 48 h, vegetative cells from the edge of the disks were sub-cultured twice consecutively for 72 h at 30° C. on T3 agar as described by Travers et al., (1987) Appl Enciron Microbiol, 53:1263-1266. At the end of each subculture, colonies were checked microscopically to verify that the cultures had sporulated and that parasporal crystals had been produced. After the second subculture, five plates of T3 agar were inoculated and allowed to grow at 30° C. for at least 96 h. After a final microscopic examination to verify sporulation and crystal production, 10 mL of sterile water was pipetted onto each of the five plates, and a sterile metal spreader was used to gently remove the colonies from the plate. The resulting spore and crystal suspension was removed from the plate using a 5 mL pipette and placed into sterile, disposable 50 mL conical centrifuge tubes.

Cry1Ab (SEQ ID NO: 2) and Cry1Ba (SEQ ID NO: 1) were selected as the individual toxins to test. The Cry1Ab (SEQ ID NO: 2) from Bacillus thuringiensis kurstaki HD1 was produced in E. coli on LB media with ampicillin (40 μg/mL) at 37° C. with shaking for 24 h, according to Geiser et al. (1986). Cry1Ba (SEQ ID NO: 1) was produced in a Bacillus thuringiensis strain in a Cry minus background (Brizzard and Whiteley, (1988) Nucleic Acids Res, 16:2723-2724), with NBY Medium (Thorne, (1968) J Virol, 2:657-662) at 30° C. with shaking at 180 rpm for 72 h or until sporulation and crystal formation was evident.

Following isolation, the strain isolates and the individual toxins had to be solubilized and activated prior to measuring their potential toxicity. Harvested spores and crystals were washed three times with 300 mM NaCl, 10 mM EDTA, pH 8.0, supplemented with PMSF 0.1 mM. Enriched Cry toxins pellets from each Bt strain tested were solubilized and activated with trypsin according to Fernandez-Luna et al. (2010), J Invertebr Pathol, 104:231-233, with minor modifications. For the solubilization, the crystals and spores were pelleted at 12,000 g for 20 min at 4° C. The pellet was resuspended and sonicated in 10 mL buffer (50 mM Tris-HCl pH 8 supplemented with Lysozyme 200 μg/mL). The pellet was sonicated three times for intervals of 10 seconds and 1 minute in ice. The crystals and spores were spun down again at 12,000 g for 20 min at 4° C. The crystals were then solubilized by resuspending the pellet in 12 mL of 50 mM sodium carbonate (pH 10.5) with 5 mM DTT and incubated for 3 hours at 37° C. with 120 rpm orbital shaking.

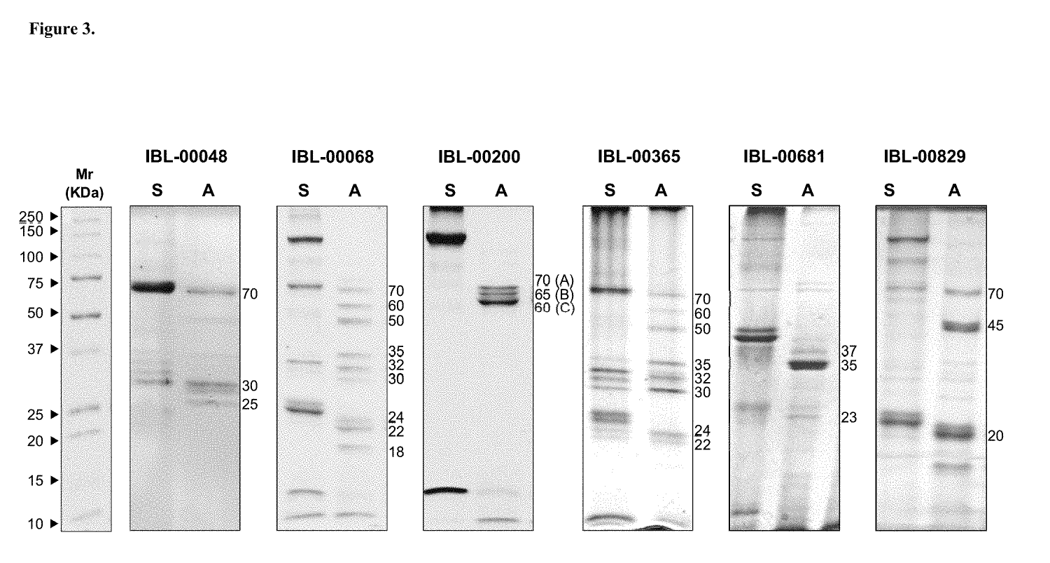

After solubilization, samples were centrifuged at 12,000 g for 10 min at 4° C. to pellet the spores and non-soluble material. The solubilized sample was dialyzed against 50 mM Tris-HCl pH 8.8 with three buffer exchanges (1 L each), and protein quantified (Bradford, (1976) Anal biochem, 72:248-254) using bovine serum albumin as standard. Due to the lack of information about ACP digestive gut proteases, which are essential for activation of Bt toxins in the insect gut, in vitro activated Cry toxins were used in ACP feeding assays. Trypsin from bovine pancreas (TPCK treated; Sigma-Aldrich, St. Louis, Mo.) was used at a 10:1 ratio (w/w) of toxin:trypsin for activation. The reaction was conducted for 1-2 h at 37° C. with 180 rpm orbital shaking. Trypsin was removed by trypsin affinity matrix (Benzamidine Sepharose 6B, GE Healthcare, Marlborough, Mass.) before use in ACP feeding assays following the protocol of the manufacturer. The toxin profile was examined by SDS-PAGE with 12% acrylamide gels. The protein concentrations of toxin samples were quantified and samples stored at −80° C. until use (FIG. 3).

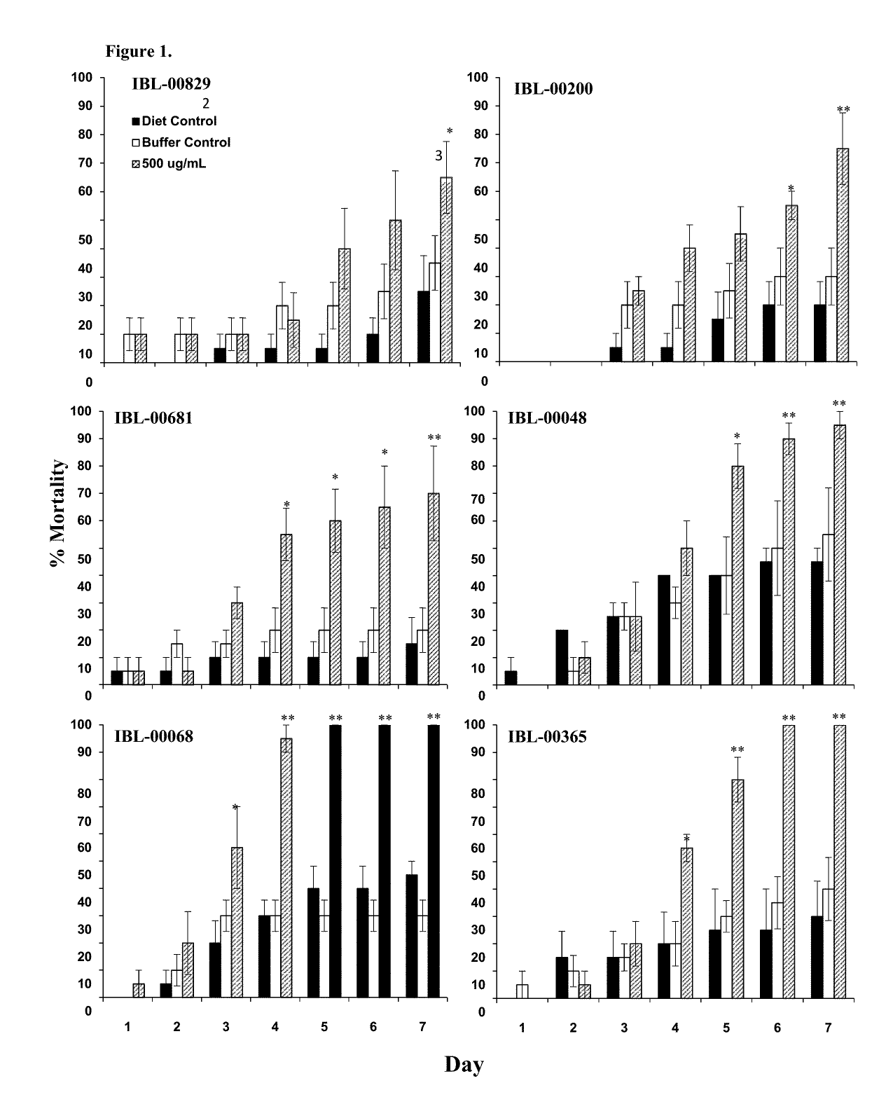

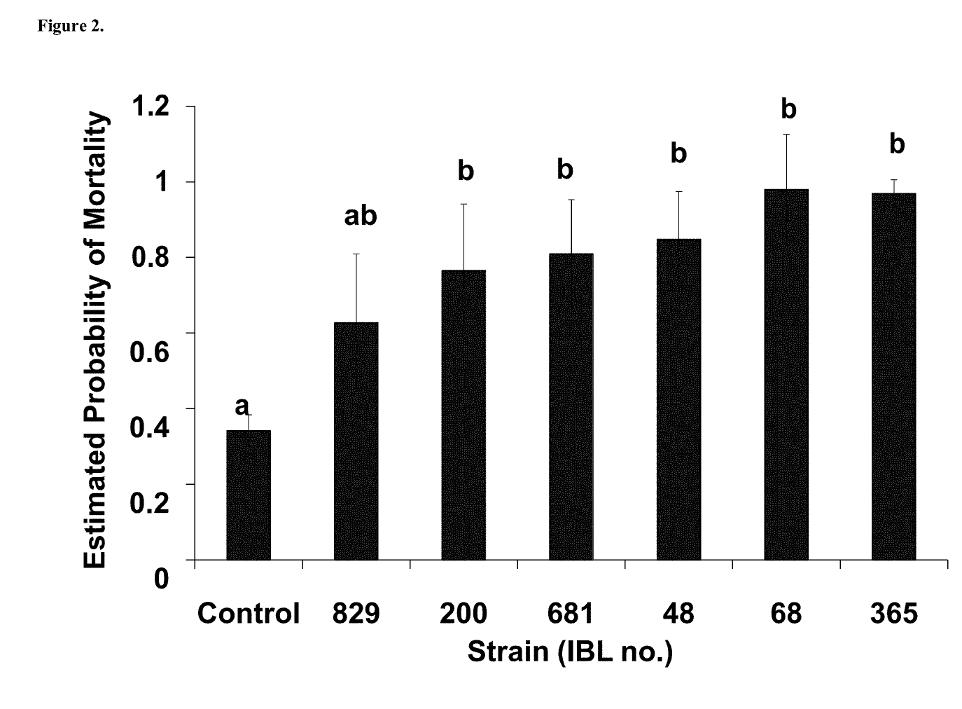

Our first objective was to identify Bt strain-derived toxins that have toxicity against ACP. To achieve this, toxin mixtures derived from each of 18 Bt strains and two recombinant Bt Cry toxins were screened in ACP bioassays using toxin preparations that had been solubilized and trypsin activated to expose ACP to activated toxins (Table 1). Of the 18 Bt isolates tested, six strains expressed toxins that were toxic to ACP (FIG. 1), the two recombinant Cry toxins −Cry4A and Cry11A were not toxic, while 12 strains lacked toxicity (Table 1). For the six isolates with significant ACP mortality compared to control treatments, a toxin dose of 500 μg/mL resulted in mortality at day 7 ranging from 50 to 100% (FIG. 1). This toxin dose was the only dose of those tested that induced significant mortality relative to control treatments. Mortality induced by toxins derived from each Bt strain (normalized with the control mortality) at day 7 was: IBL-00068, 70%; IBL-00365, 60%; IBL-00681, 50%; IBL-00200, 45%; IBL-00048, 40%; IBL-00829, 30%. The estimated probability of mortality at day 7 shows significantly increased probability for these six treatments compared to the control (logistic regression analysis; FIG. 2).

### Screening of Bt Accessions for Toxicity to Adult ACP

Relative toxicity of individual strains and endotoxins was investigated by comparing mortality rates of ACP feeding on different concentrations of Bt in a liquid diet to mortality of ACP feeding control diet. The ACP were obtained from a colony previously described (Hall et al., (2010) Ann Entomol Soc Am, 103: 611-617; Hall et al., (2015) Crop Protection, 72:112-118). The psyllids were continuously reared in a greenhouse on Citrus macrophylla Wester, a genotype favored by ACP for colonization (Westbrook et al., (2011) Hortscience, 47:997-1005). The colony was maintained using procedures similar to those described by Skelley and Hoy, (2004) Biol Control, 29:14-23, and tested quarterly to ensure that the colony remained HLB-free.

The initial base diet for adult psyllids (Hall et al. 2010) consisted of sucrose (30%), yellow food coloring (0.4%), and green food coloring (0.1%) in tap water, with diet autoclaved after mixing. Addition of trypsin-activated Bt toxins to this diet at concentrations of 25, 50, 100 and 500 μg/mL resulted in precipitation. Subsequently, food coloring was excluded from the psyllid diet and a sachet of green diet placed behind the sachet of clear diet with Bt to attract ACP to the diet.

Five adult psyllids (≤7 days-old) per plastic vial were subdued by cooling at 5° C. Vials were removed from the refrigerator one at a time, and psyllids placed into the feeding chamber dish in a clean air hood. A 4.8×2.5 cm piece of Parafilm® membrane was stretched across the dish above the psyllids. A slight indentation of the membrane was created with a sterile gloved finger in the center of the membrane, and 0.5 mL of the liquid diet was pipetted into this depression. A second membrane was then stretched across the first, sandwiching the diet. The sachet of green diet was then placed on top of the sachet of clear diet. Feeding chambers were then placed in a growth chamber at 25° C., 14:10 h L:H and 75% RH.

Samples of activated toxin mixtures from individual Bt strains and of individual purified toxins were tested for toxicity, with five adult psyllids per feeding chamber and four feeding chambers (technical replicates) per toxin concentration (n=20 psyllids total per treatment). Control dishes included sugar diet only (Diet Control) and sugar diet with the same volume of 50 mM Tris-HCl pH 8 added as for the toxin samples (Buffer Control), to control for possible buffer effects on psyllid survival. Between one and four biological replicates were conducted for each bioassay. Mortality was scored daily. Bioassays with excessive control mortality were eliminated. Control mortality of 30% or lower was considered acceptable, unless mortality in the test treatment was >2× (p<0.05; Student's t test) the mortality in the control treatment.

On the third, seventh and eleventh day of each bioassay, the relative quantity of ACP excretions (honeydew) present in each feeding chamber was rated (0: none; 1: <5 droppings; 2: ≥5 droppings). This analysis was done for one to three of the biological replicates with a minimum of 20 ACP scored per treatment.

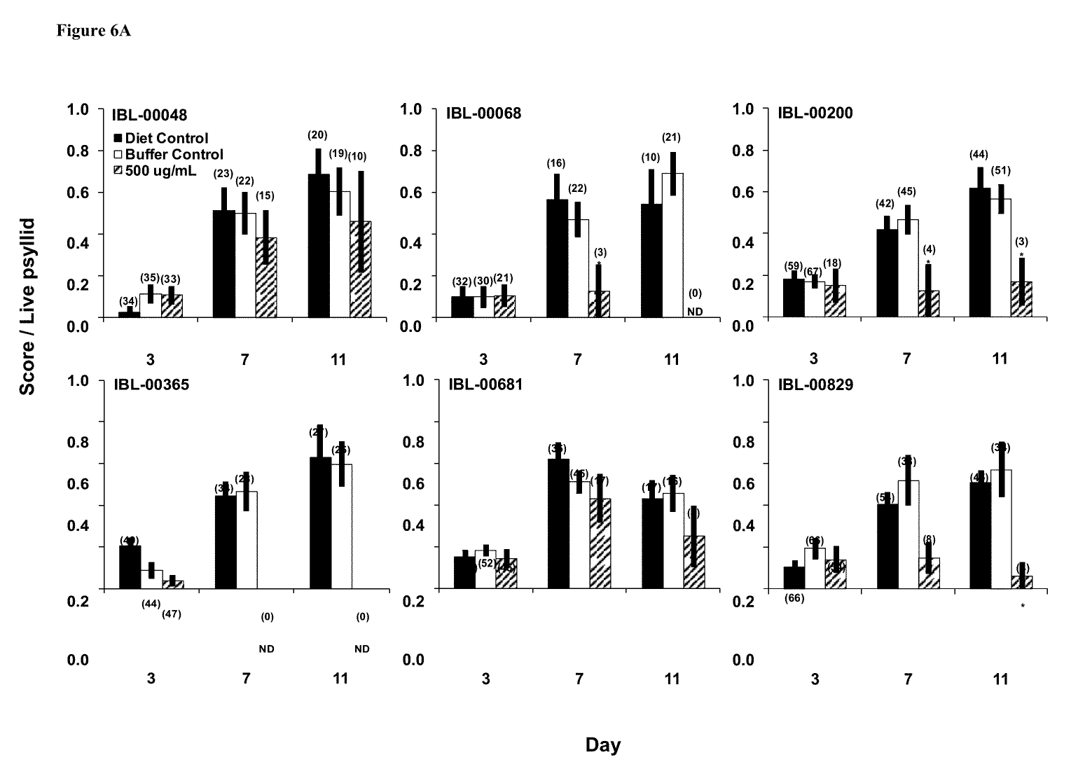

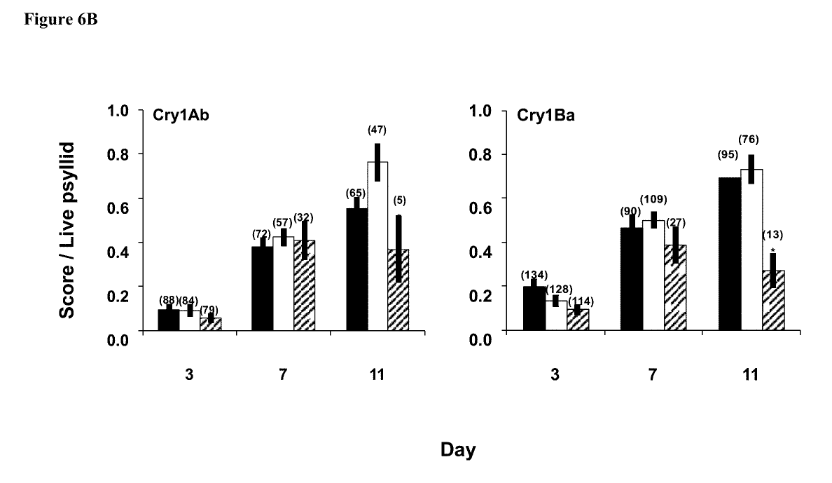

The insecticidal mechanisms that result in the toxicity of Cry proteins are incompletely understood. However, the intoxication effects of Cry toxins include disruption of the midgut epithelium resulting in gut paralysis, cessation of feeding, starvation and eventual death (Schnepf, et al. (1998)) The production of honeydew, which is produced as psyllids feed (Ammar et al. (2011), Fla Entomol, 94:340-342) was monitored as an indicator of feeding cessation. Based on this assay, adult ACP stopped feeding on some Bt strains (FIG. 6A). Specifically, ACP fed on toxins derived from strains IBL-00068, -00200, and -00829 at a dose of 500 μg/mL produced significantly fewer excretions compared to the buffer control diet after 7 days of exposure (Dunn's test; p<0.05. All insects in the IBL-0365 bioassay were dead by day 7 such that data for this strain could not be included in the analysis, FIG. 6A). This result suggests that mortality recorded in these assays resulted from cessation of feeding induced by the action of Cry toxins.

### Toxicity of Individual Toxins to ACP

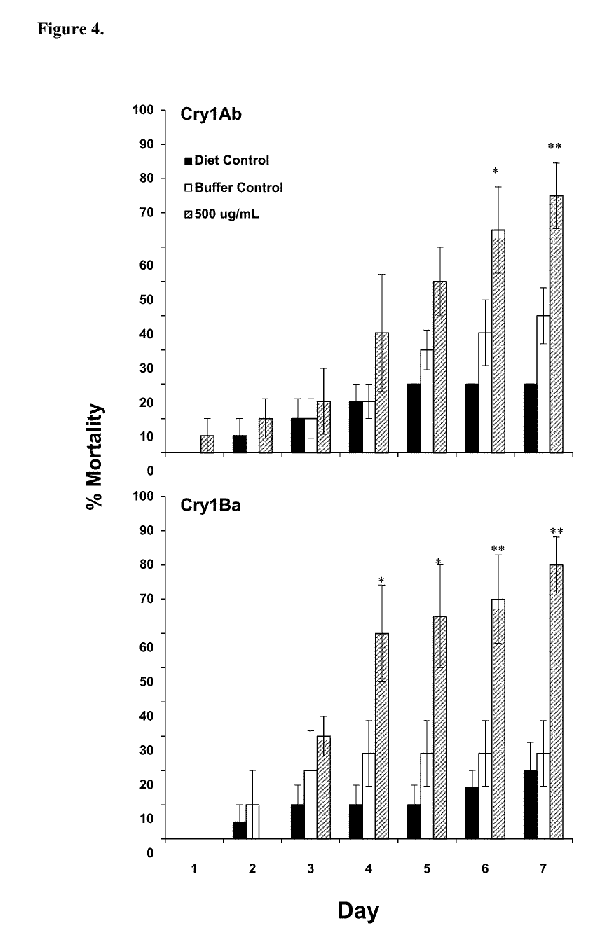

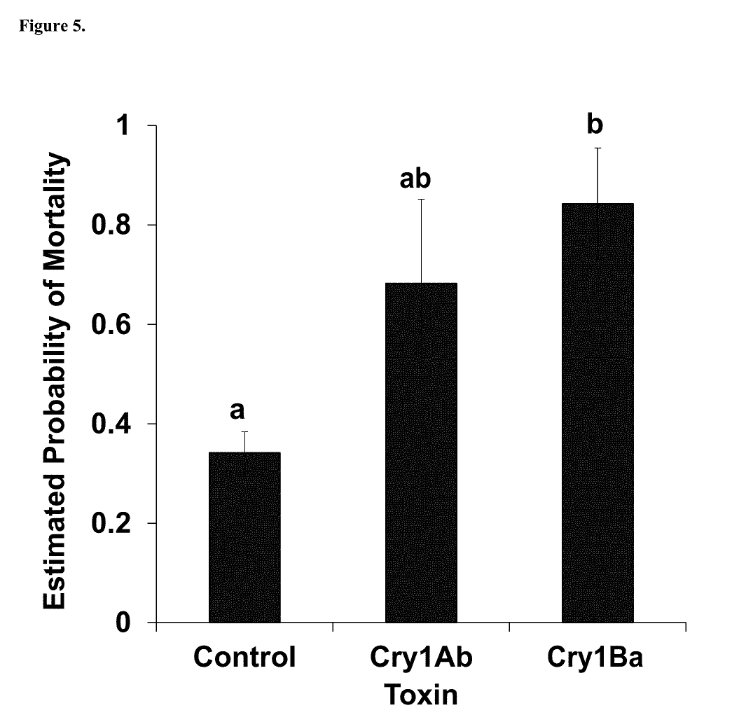

To understand the contribution of individual toxins derived from IBL-00200 to the ACP toxicity induced by this strain, we tested Cry1Ab (SEQ ID NO: 2) and Cry1Ba (SEQ ID NO: 1; which has 83% amino acid identity to Cry1Bb (SEQ ID NO: 3); it cannot be assumed that Cry1Ba and Cry1Bb have the same impact on ACP however). Bioassays showed that both Cry1Ab and Cry1Ba have basal levels of toxicity against ACP (FIGS. 4, 5). Both strains are toxic at 500 μg/mL: Cry1Ba resulted in 60% mortality by day 4, while Cry1Ab resulted in 60% mortality by day 6 (FIG. 4). For comparison, the IBL-00200-derived toxin mixture resulted in 42% mortality by day 4 and 55% mortality by day 6 (FIG. 1). A significant reduction in production of honeydew was noted for ACP fed on Cry1Ab and Cry1Ba after 11 days of exposure when compared to the Buffer control (Dunn's test; p<0.05; FIG. 6B). This result, with significant differences recorded at 11 days rather than at 7 days as for strain-derived toxin mixtures, suggests that the combined action of toxins derived from IBL-00200 resulted in feeding cessation more rapidly than the individual components tested.

### Identification of Toxins Produced by IBL-00200

Bt strain IBL-00200 was among the strains identified as having toxicity to ACP. The solubilized and trypsin-activated toxins were separated by SDS-PAGE (12% gel, FIG. 3), protein bands isolated and treated with trypsin for protein identification through LC-MS/MS. The translated IBL-00200 genome was used as reference for identification of proteins based on trypsin peptide profiles generated by MS/MS, using Thermo Scientific Proteome Discoverer software. Maximum likelihood analysis of protein sequences was performed with MEGA 6 (Tamura et al. 2013) using the default parameters.

Solubilization and activation of Bt strain-derived toxins generated different proteolytic profiles, with a total of ten distinct proteolytic profiles identified from the toxin strains examined. Profiles of trypsin-activated toxins are shown for strains with toxicity to ACP (FIG. 3). The pro-toxin protein profile (i.e. soluble protein) of the majority of the Bt isolates produced bands of approximately 25, 40, 75 and/or 120 kDa. The molecular mass of the trypsin-activated toxins ranged from 20 kDa to 70 kDa, suggesting a diverse composition of Cry and possibly also Cyt toxins.

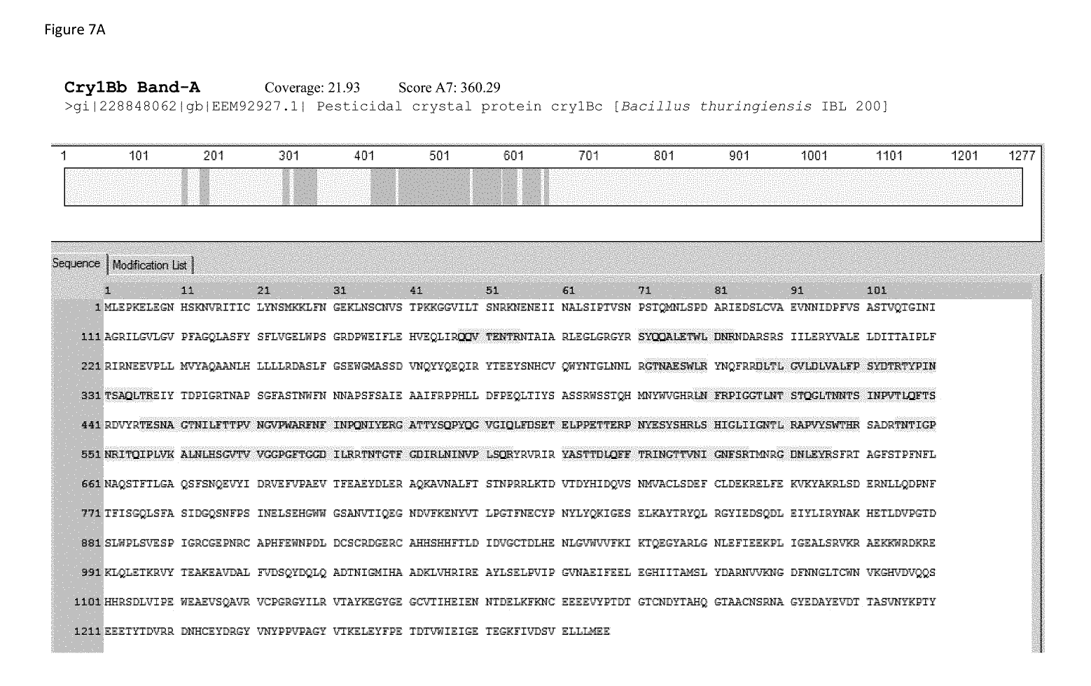

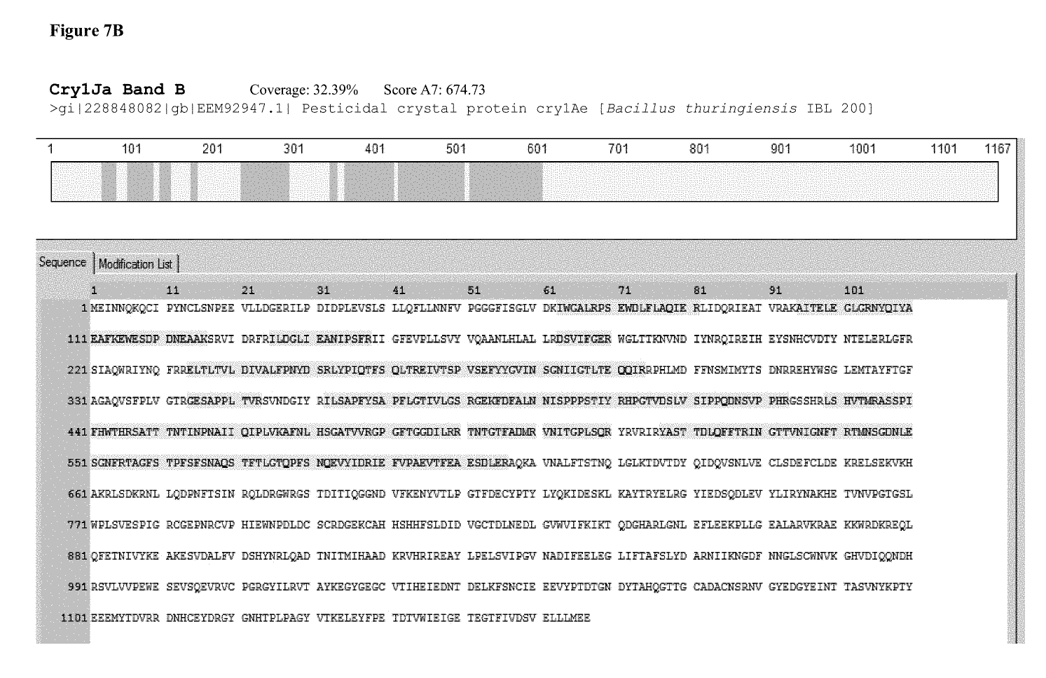

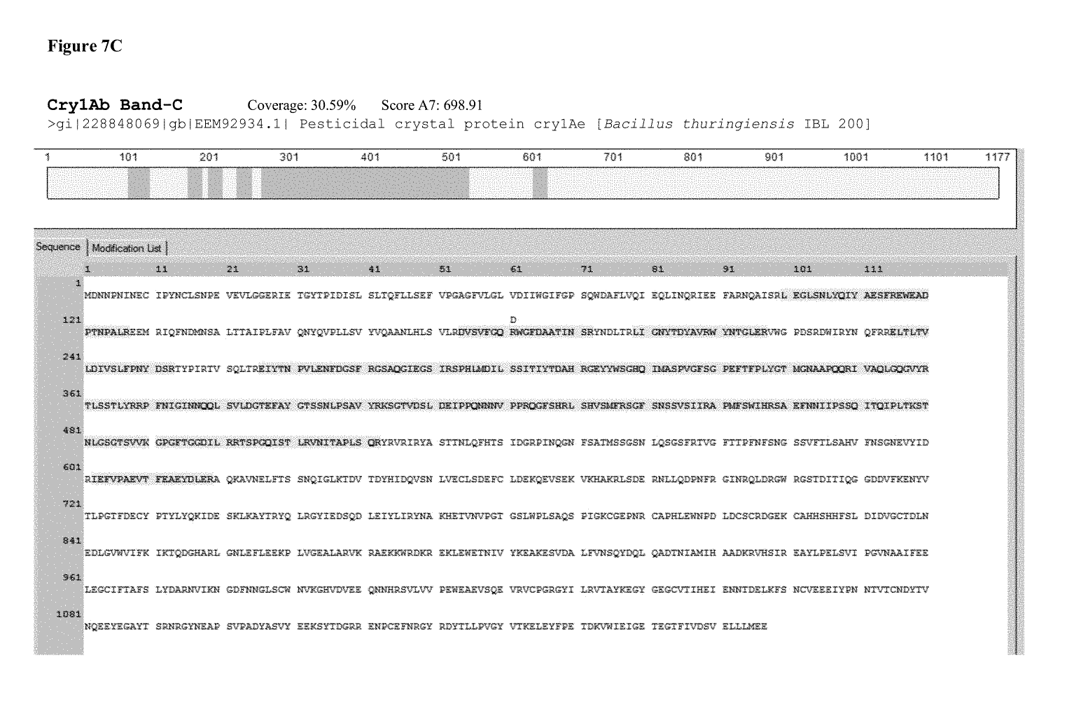

Strain IBL-00200 was selected for analysis of the toxicity of individual toxins produced by that strain. The proteolytic profile after trypsin treatment shows three bands of different molecular mass (A, B, C; FIG. 3). These bands were cut from the gel and submitted for peptide sequencing and toxin identification (FIG. 7A-7C). Peptide sequences obtained from the bands were found to be consistent with the translated products of three genes found on a 55 kb fragment of the partially assembled IBL-00200 genome (GenBank: ACNK01000108.1). Peptides obtained from bands A, B, and C were consistent with GenBank proteins EEM92927.1 (SEQ ID NO: 3), EEM92947.1 (SEQ ID NO: 4), and EEM92934.1 (SEQ ID NO: 2) respectively, all of which are encoded on the 55 kb fragment (Table 2). EEM92927.1 was annotated as a Cry1Bc, while both EEM92947.1 and EEM92934.1 were annotated as Cry1Ae. However, maximum likelihood analysis of the predicted protein sequences with those of holotype cry1 toxins (http://www.btnomenclature.info), reveal that EEM92927.1 (SEQ ID NO: 3) is most closely related to Cry1Bb, while EEM92947.1 (SEQ ID NO: 4) is most closely related to Cry1Ja, and EEM92934.1 (SEQ ID NO: 2) to Cry1Ab. Thus, the tryptic fragments A, B, and C observed in FIG. 3 are due to proteins highly similar to Cry1Bb, Cry1Ja, and Cry1Ab, respectively. It is notable that although the IBL-00200 genome appears to include a number of other cry genes, these three were the only ones detected in the parasporal crystal (Table 3).

### Transmission Electron Microscopy.

Adult ACP were fed by membrane feeding on artificial diet in 30% sucrose-TRIS with trypsin activated toxin extract of IBL-00200 (500 μg/mL), Cry1Ba (SEQ ID NO: 1) (500 μg/mL) or Tris buffer alone added. Twenty insects per treatment were fed for 48 hr in a growth chamber at 25° C., 14:10 h L:H and 75% RH, with two replicate assays. ACP from all replicates were pooled. The distal abdominal segments, head and legs were removed and torsos then immediately fixed (2% paraformaldehyde, 2.5% glutaraldehyde, 0.05 M cacoldylate buffer, pH 7.1) for 48 hours at 4° C. Samples were washed in buffer and then fixed in 1% osmium tetroxide in 0.1 M cacodylate buffer for 1 hour. The samples were then en-bloc stained with 2% uranyl acetate for 2 hours, dehydrated in a graded ethanol series, cleared with ultra-pure acetone, infiltrated and embedded using a modified EPON epoxy resin (Embed 812; Electron Microscopy Sciences, Ft. Washington, Pa.). Resin blocks were polymerized for 48 hours at 70° C. Thick and ultrathin sections were made using a Leica UC6 ultramicrotome (North Central Instruments, Minneapolis, Minn.). Ultrathin sections were collected onto copper grids and images were captured using a JEM 2100 200 kV scanning and transmission electron microscope (Japan Electron Optic Laboratories, LLC, Peabody, Mass.).

Ingestion of activated toxins derived from IBL-00200, and of activated Cry1Ba (SEQ ID NO: 1) resulted in widespread damage to the ACP midgut epithelium. While gut microvilli in control insects appeared densely packed and regular in organization, microvilli had disintegrated in ACP fed on IBP-00200-derived toxins. The microvilli of insects fed Cry1Ba were disorganized, and detached from the cell surface and cell debris was evident in the gut lumen.

### Statistical Analysis

Statistical significance was assessed using one-way ANOVA and Student's t tests to evaluate differences between groups, with the use of GraphPad Prism version 5.00 for MacOSX (GraphPad Software, San Diego, Calif.). A p-value of less than 0.05 was considered statistically significant unless otherwise indicated. If an overall significance was detected, Tukey's multiple range tests were performed. All data were analyzed prior to statistical analysis to meet the homoscedasticity and normality assumptions of parametric tests. For each dose level, logistic regression (Proc Logistic; SAS version 9.4; SAS Institute, Cary, N.C.) was used to examine whether there were significant differences in mortality patterns between the strains and control. A fixed effect for block was included in the model so that we can compare treatments after accounting for environmental conditions that may have affected mortality.

Overdispersion was detected for the 500 ug/mL dose (Deviance/DF=1.5167, p=0.0010) and accounted for. Mortality of 100% was observed for at least one of the strains, leading to issues with maximum likelihood estimation. We instead use Firth's penalized likelihood for estimation. FIGS. 2 and 5 show the estimated probability of mortality and standard errors for each treatment.

For calculation of significant differences in honeydew excretion between test and Buffer control treatments at different times post-exposure, non-parametric tests were employed. Groups were analyzed using the Kruskal-Wallis test and major differences observed were examined further using Dunn's two-tailed nonparametric multiple comparison test (GraphPad Prism version 5.00 for MacOSX). A p-value of less than 0.05 was considered statistically significant.

TABLE 1
Bt strains and individual toxins tested for toxicity against ACP.
Activated and purified toxins from each strain, or individual toxins
were tested in bioassays with adult ACP as described.

| Toxic | Non-toxic |
|---|---|
| IBL-00048* | IBL-00024 |
| IBL-00068 | IBL-00055 |
| IBL-00200 | IBL-00071 |
| IBL-00365 | IBL-00090 |
| IBL-00681 | IBL-00098 |
| IBL-00829 | IBL-00192 |
| Cry1Ab (SEQ ID NO: 2) | IBL-00217 |
| Cry1Ba (SEQ ID NO: 1) | IBL-00438 |
| | IBL-00937 |
| | IBL-01306 |
| | IBL-01313 |
| | IBL-03792 |
| | Cry4A |
| | Cry11A |

*Single biological replicate.

TABLE 2
Cry toxins expressed by strain IBL-00200 based on MS/MS
data generated. Score A4: sum of the scores of the individual
peptides from the Sequest HT search (calculated by: 0.8 +
peptide charge × peptide relevance factor). Data
were analyzed by Proteome Discoverer software V2.1 for
Windows (Thermo Scientific, Waltham, MA).

| | | Identified | | |
|---|---|---|---|---|
| Band | Accession Number | Cry toxin | Coverage (%) | Score A4 |
| A | EEM92927.1 (SEQ | Cry1Bb | 21.93 | 360.29 |
| | ID NO: 3) | | | |
| B | EEM92947.1 (SEQ | Cry1Ja | 32.39 | 674.73 |
| | ID NO: 4) | | | |
| C | EEM92934.1 (SEQ | Cry1Ab | 30.59 | 698.91 |
| | ID NO: 2) | | | |

TABLE 3
Toxins encoded by Bt strain IBL-00200 according to the genome
sequence (Bowen et al., (1998) Science, 280: 2129-2132)
and de novo identification of the expressed toxins under
sporulation conditions based on the Bt toxin nomenclature
database - http://www.btnomenclature.info.

| | | Expression in IBL-00200 | |
|---|---|---|---|
| Accession | Previous | and designation based on | |
| numbers | designation | Bt database | Locus_tag |
| EEM93105.1 | cry11Bb | ND | |
| EEM93049.1 | cry2Ad | ND | |
| EEM93050.1 | cry2Ad | ND | |
| EEM93051.1 | cry1Ae | ND | |
| EEM93055.1 | cry2Ad | ND | |
| EEM93056.1 | cry1Ae | ND | |
| EEM92924.1 | cry1Ae | cry1Hb | |
| EEM92927.1 | cry1Bc | cry1Bb (SEQ ID NO: 3) | bthur0013_56890 |
| EEM92934.1 | cry1Ae | cry1Ab (SEQ ID NO: 2) | bthur0013_56960 |
| EEM92941.1 | cry1Bc | ND | |
| EEM92947.1 | cry1Ae | cry1Ja (SEQ ID NO: 4) | bthur0013_57090 |
| EEM92952.1 | cry1Bc | ND | |
| EEM92953.1 | cry1Ae | cry1Da | |
| EEM92570.1 | cry8Ba | ND | |

### Example 2

Identification of Asian Citrus Psyllid, Diaphorina citri (Hemiptera) Gut Binding Peptides and Creation of Gut Peptide Binding Peptide Fusion Proteins to Increase Binding of Conjugates to Insect Guts

In this example, we screened multiple peptides that could bind to the gut of insects, specifically to the gut of the Asian citrus psyllid (ACP). An expression of vector combing these peptides with a linker and a reporter protein was then created and tested to show it bound to ACP gut and that this binding was due to the gut binding peptide.

### Insects

Asian citrus psyllid, ACP (Hemiptera), were obtained from USDA ARS, Fort Pierce, Fla. (Hall et al. (2010); Hall et al. (2015)). Adults (5 to 7 days old) were used for toxicity assays and for BBMV preparation. Psyllids were continuously reared in a greenhouse on Citrus macrophylla Wester, a genotype favored by ACP for colonization (Westbrook et al. (2011)). The colony was maintained as described by Skelley and Hoy (2004) and tested quarterly to ensure that the colony remained HLB-free.

### Phage Selection and Identification of ACP Gut Biding Peptides

A phage display library (Ph.D.-C7C, New England Biolabs) based on the M13 phage vector modified for display of a randomized segment consisting on a heptapeptide as N-terminal fusions to the minor coat protein pIII was used. The randomized segment of the library is flanked by a pair of cysteine residues resulting in displayed peptides being presented to the target as loops. The library was amplified in E. coli host strain ER2738 and phage were precipitated using 20% PEG 8000 (Sigma, St. Louis, Mo.) and 2.5 M NaCl. Methods used for preparing cells for infection, amplifying and preparing phage libraries were as recommended by the supplier (New England Biolabs, Ph.D.-C7C). The phage concentration was determined by titration.

For selection of phage that bound to the ACP gut, seven adult psyllids (≤7 days-old) were starved at 4° C. overnight (16 h), and then fed with 100 μl of PBS containing 30% sucrose and ˜1×10¹⁴ pfu of f88.4 phage by membrane feeding at room temperature for 4 to 16 h. The guts of 30-50 ACP were isolated after feeding (Liu et al., (2006) Advances in Virus Res, 68:427-57) and suspended in 100 μl PBS (100 mM, pH 7.0) containing 1% BSA. The gut tissues were gently ground by using a Kontes pellet pestle (Fisher Scientific, Pittsburgh, Pa.) and the tissue suspensions spun at 1000×g in a bench top centrifuge for 1 min. The supernatant was removed and pellets were resuspended in 500 μl of PBS, 1% BSA to remove unbound phage. The resuspensions were centrifuged as previously, and the washing step was repeated once. The bound phage were eluted by adding 300 μl of elution buffer (50 mM glycine-HCl, pH 2.2, 1 mg/mL BSA) and rotated gently in a Labquake™ Shaker (Barnstead/Thermolyne, Dubuque, Iowa) for 5 to 8 min at room temperature, and then briefly centrifuged at 1000×g. The supernatants were transferred to a 1.5-mL tube and neutralized by adding 8 μl of 2 M Tris-base (pH 9.1). Twenty microliters of the eluted phage were used for titration to estimate the number of recovered phage from the biopanning process. The remainder of the eluted phage was amplified immediately in ER2738 cells (New England Biolabs, Ph.D.-C7C). The enriched phages were titrated and used for the next round of biopanning. The biopanning process was repeated three times, and the entire phage display library screen was repeated twice.

After the third round of selection, eluted phage were plated on to LB/IPTG/Xgal plates. Individual randomly selected bacterial colonies were cultured and used for isolation of phage DNA following standard phage DNA purification procedures (Sambrook and Russell, 2001). The sequencing primer (5′-CCCTCATAGTTAGCGTAACG-3′) (96 sequencing primer) (SEQ ID NO: 13) was used for sequencing to determine the predicted amino acid sequence of inserts in the selected phage.

Three sequences were enriched after three rounds of biopanning against the ACP gut. Peptide-18 was obtained in the second round of biopanning (see Table 4 for full results).

| | | | |
|---|---|---|---|
| Peptide 12 | (S K H S L S Q) | 1/14 | (SEQ ID NO: 5) |
| Peptide 15 | (T T K L P N S) | 9/14 | (SEQ ID NO: 6) |
| Peptide 18 | (E T P S R A R) | 2/26 | (SEQ ID NO: 7) |
| Peptide 22 | (N N S G K Q L) | 2/14 | (SEQ ID NO: 8) |

### Production of Peptide-mCherry Fusions

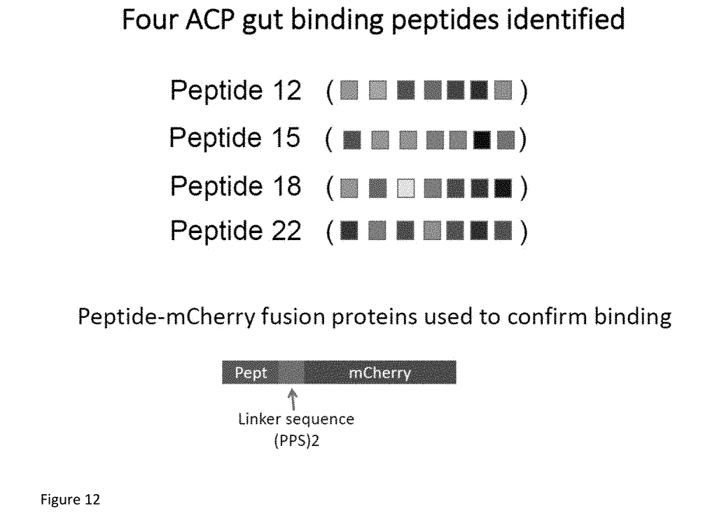

The pBAD/His B expression vector was used for expression of peptide-mCherry fusions with His tags (Invitrogen, San Diego, Calif.). Four gut binding peptides (pept-12, pept-15, pept-18, pept-22) and a nonbinding Control peptide (C) were fused at the N-terminus of mCherry and inserted into pBAD/His B using molecular standard procedures (see Table 5 for construction primers, FIG. 12).

For expression of fusion peptide-mCherry competent DH5a cells were transformed with the respective plasmid. Cells were cultured in 50-mL low-salt LB medium containing ampicillin (100 μg/mL) in a 500-mL flask and shaken in an orbital shaker at 250 rpm (37° C.) until the OD₄₅₀ reached 0.4 to 0.5. L-(+)-arabinose.

### Toxin Blots

Brush border membrane vesicles (BBMV) isolated from 7 day old ACP adults were prepared as reported previously (Nielsen-LeRoux and Charles, (1992) Eur J Biochem, 210:585-590). For the first dimension, BBMV proteins (50 μg) were precipitated using the 2D Clean Up Kit (GE Amersham Biosciences) and resuspended in hydration buffer [8 M urea, 2% w/v CHAPS, 15 mM DTT and 0.5% v/v IPG buffer pH 3-10 (GE Amersham Biosciences)]. Isoelectric focusing was performed by use of IPG strips (7 cm, pH 3-10; GE Amersham Biosciences) that were hydrated for 2 h and focused for 8 h at 50 V, 1 h at 500 V, 1 h at 1000 V, 2 h at 8000 Vat 20° C. under mineral oil. The IPG strips were first incubated for 15 min in equilibration buffer I (6 M urea, 30% glycerol, 2% SDS and 1% DTT (w/v) in 0.05 M Tris-HCl buffer pH 8.8), and then 10 minutes with equilibration buffer II (6 M urea, 30% glycerol, 2% SDS and 4% iodoacetamide (w/v) in 0.05 M Tris-HCl buffer, pH 8.8). After equilibration, strips were electrophoresed in a 2D PAGE gradient precast gel with IPG well (Bio-Rad; Catalog #456-9031) for the second-dimension separation, by running at 180 V, at 4° C. for 1 h. The proteins were electroblotted to PVDF Immobilon membranes (Amersham) at 350 mA by 45 min using transfer buffer (20% methanol, 25 mM Tris-base, 192 mM glycine). After blocking the membrane with milk 5% in PBS containing 0.2% Tween-20, the membranes were incubated for 1 h with 10 nM of the mCherry fusion peptide. Blots were washed three times using PBS containing 0.2% Tween-20. The bound fusion peptide was detected by the use of mCherry antibody and further detected with anti-rabbit peroxidase conjugate and developed with SuperSignal chemiluminescence substrate (Pierce).

### Cry1Ab and Cry1Ba Toxins Sites for Engineering

The Cry1BaA4 was modeled with Pymol based on Cry1Aa (Li, et al, (1991) Nature, 353:815-821) with which it shares 59% identity.

The N terminal of Domain I has previously been modified by Mehlo, et al., (2005) Proc Natl Acad Sci USA, 102:7812-7816, showing a toxicity increase. Furthermore, the trypsin activated N-terminal has been reported between α1-α2 (Cry1Ab: Gomez, et al., (2002) FEBS Lett, 513:242-246) in the 64 VLGVPF 69 residues (SEQ ID NO: 14). Based on these facts, in the present work we replaced the corresponding region in each Cry1Ab (SEQ ID NO: 2) and Cry1Ba toxins (SEQ ID NO: 1).

Domain II has been clearly shown to be involved in the toxin binging to the receptors present in the midgut cells. In particular, the Cry1Ac loop 2 (Jenkins, et al., (2000) J Biol Chem, 275:14423-14431) was sequence residues 395 GVLLWGIYL 403 (SEQ ID NO: 15) was demonstrated to bind the receptors. On the other hand, Cry1Ac loop3 was also involved in receptor binding (Jenkins, et al., 2000). Domain III has been involved in receptor binding. It was demonstrated that Cry1Aa bind to receptors through β-16 and β-22 (Atsumi, et al., (2005) Appl Environ Microbiol, 71:3966-3977).

### Cry1Ba Toxic Core Identification and Cloning

Based on structure prediction, protein molecular weight after trypsin treatment and N-terminal sequencing of the toxin after trypsin treatment the N-terminal and C-terminal regions of the Cry1Ba toxic core were identified. The N-terminal sequencing results show EDSLCIAEGNNIDPFVSAST (SEQ ID NO: 9) as the N-terminal amino acid sequence while the C-terminus corresponds to the amino acid sequence EIIPVTAT (SEQ ID NO: 10). The protein fragment corresponding to the region with these boundaries has 608 residues with an estimated molecular mass of 68.5 kDa. The corresponding 12 nucleotide sequence for the N terminal sequence is 91 GAG GAT AGC TTG 102 (SEQ ID NO: 11) and the 12 nucleotide sequence for the C terminus is 1891 GAA ATT ATT CCA 1902 (SEQ ID NO: 12).

### Pulldown Assays and Homologous Competitions

Binding of fusion-peptide to ACP brush border membrane vesicles (BBMV) in the presence or absence of synthetic peptide previously incorporated into the membrane. The ACP BBMV were preincubated 1 h at 25° C. with (A) or without (B) different excess of synthetic peptide in 100 mL of binding buffer, washed three times by centrifugation, and a membrane pellet was used afterward to analyze the binding of 10 nM fusion peptide (1 h at 25° C.) performed in the presence of different molar excess of unlabeled synthetic peptide as reported in Lee et al., (1995), Appl Environ Microbiol, 61:3836-42. Unbound fusion peptide was removed by centrifugation. Membrane pellet was blotted to Hybond-ECL nitrocellulose membranes. The biotinylated toxin that remained bound to the vesicles was visualized by incubating with antibody against mCherry and later with antibody against rabbit-peroxidase conjugate (1:4,000 dilution) for 1 h and developed with SuperSignal chemiluminescence substrate (Pierce).

We performed 2D-ligand blot binding assays of brush border membrane vesicle (BBMV) proteins. Fusion peptide-15 bound three proteins of approximately 50, 37 and 25 kDa, all with a pI of ˜9 (data not shown).

Pore formation in insect midgut cells involves binding to the membrane and insertion of Bt toxins into the membrane, which have been recognized as reversible and irreversible steps of the mechanism of action of the toxins respectively (Liang et al., (1995) J Biol Chem, 270:217-222). In this context, we expect to enhance the reversible step by means of providing a peptide sequence that has been selected by binding in the ACP gut.

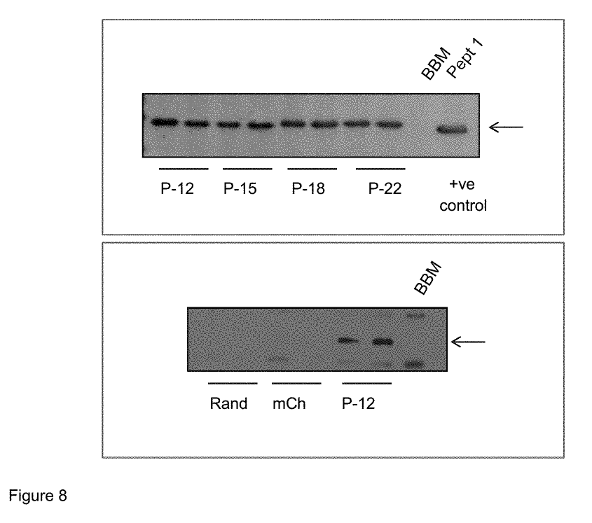

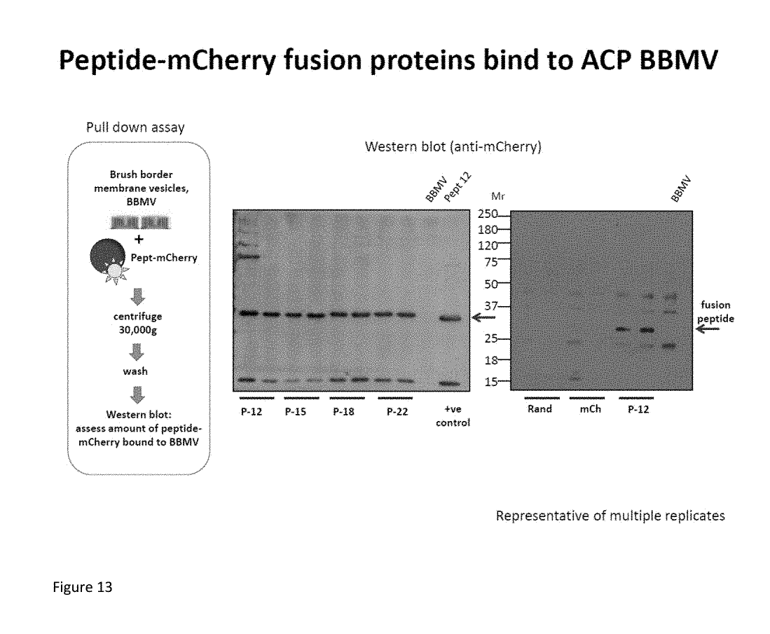

Pull down assays of peptide-mCherry fusions with ACP BBMV were used to confirm binding of peptides to BBMV proteins (FIGS. 8 and 13). A randomly selected peptide (Rand) and mCherry alone were used as negative controls in this assay. All peptides were pulled down with BBMV in this assay.

Confirmation of Fusion Peptide Binding to the ACP Gut and Selection of Fusion Peptide 15 as a Peptide that Binds Specifically

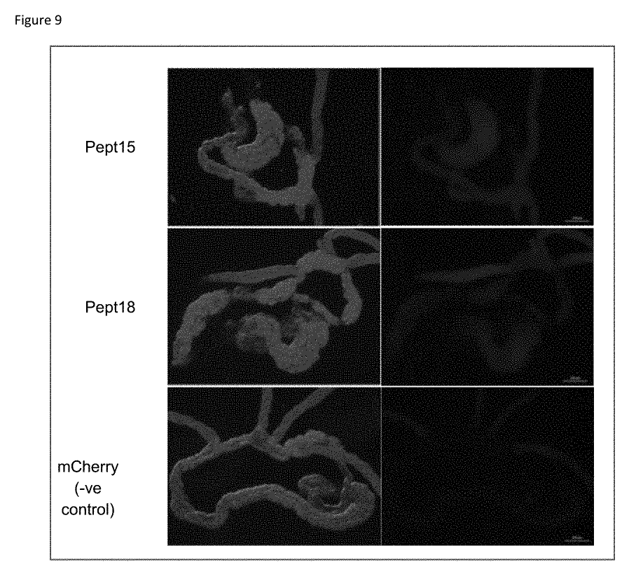

To test whether the peptides selected from the biopanning procedure and the control peptide bound to the ACP gut and whether the fusions would bind the ACP microvillae, the peptide-mCherry fusions were fed to prestarved ACP (10 psyllids per membrane feeding sachet) and the psyllids subsequently examined for red fluorescence using a fluorescence microscope (FIG. 9). Fusion proteins (1 μg) were resuspended in 100 μl of 30% sucrose in Tris 50 mM, and fed to adults by membrane feeding for 16 h. Dissected guts were then observed under a compound microscope (Zeiss Axioplan II fluorescence microscope). The gut contents were removed by washing with PBS. Images were recorded under bright field as well as with an excitation at ˜560 nm and emission at ˜610 nm light using a Zeiss Axiocam digital camera.

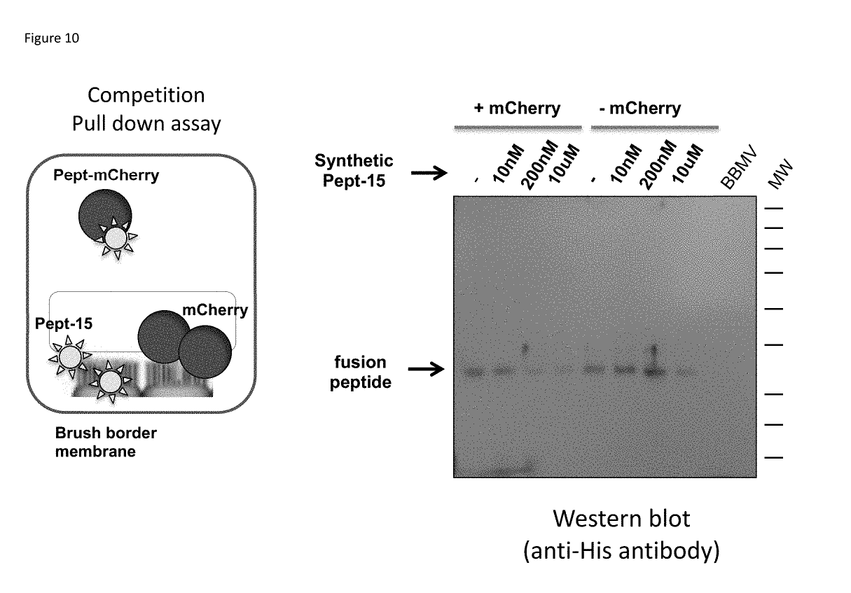

Having confirmed binding of peptide-mCherry fusion proteins by pulldown assay (FIGS. 8 and 13), competition assays were conducted with increasing amounts of synthetic peptide in the presence or absence of mCherry (FIG. 10). These assays showed that only peptide 15 (SEQ ID NO: 5) bound to ACP BBMV proteins specifically. Hence, peptide 15 was selected for use in modification of selected toxins to target ACP.

### Cry Toxin Engineering

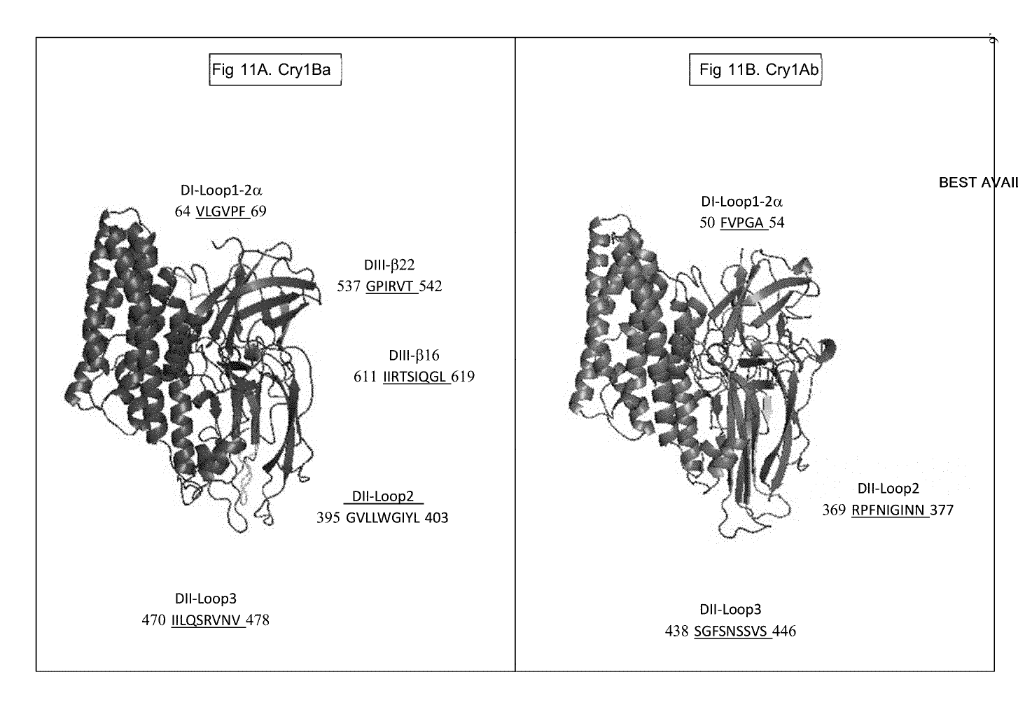

The structures of Cry1Ab (SEQ ID NO: 2) and Cry1Ba (SEQ ID NO: 1) were modeled using PyMol (FIGS. 11A-11B). Residues corresponding to the site of trypsin cleavage in Domain I and loops 2 and 3 of Domain II were determined. These sites, along with others as indicated will be used as sites for introduction of peptide 15 sequence into the toxin.

TABLE 4
Selected peptides through biopanning of PhD
library. Peptides chosen for further
experimentation are highlighted.

| Round of | | Enrich- | Percen- | Peptide |
|---|---|---|---|---|
| biopanning | Sequence | ment | tage | name |
| second | TTKLPNS | 12/26 | 46.15% | Pept15 |
| | (SEQ ID NO: 5) | | | |
| | NNSGKQL | 4/26 | 15.38% | Pept22 |
| | (SEQ ID NO: 8) | | | |
| | ETPSRAR | 2/26 | 7.69% | Pept18 |
| | (SEQ ID NO: 7) | | | |
| | NRTVPNL | 1/26 | 3.8% | |
| | (SEQ ID NO: 16) | | | |
| | KGPLPGQ | 1/26 | 3.8% | |
| | (SEQ ID NO: 17) | | | |
| | MRFPGDL | 1/26 | 3.8% | |
| | (SEQ ID NO: 18) | | | |
| | SHPHLKT | 1/26 | 3.8% | |
| | (SEQ ID NO: 19) | | | |
| | NIKSSHV | 1/26 | 3.8% | |
| | (SEQ ID NO: 20) | | | |
| | ITRTSHT | 1/26 | 3.8% | |
| | (SEQ ID NO: 21) | | | |
| | MSVSTRD | 1/26 | 3.8% | |
| | (SEQ ID NO: 22) | | | |
| third | TTKLPNS | 12/26 | 46.15% | Pept15 |
| | (SEQ ID NO: 5) | | | |
| | NNSGKQL | 4/26 | 15.38% | Pept22 |
| | (SEQ ID NO: 8) | | | |
| | KTSLHHM | 1/14 | 7.14 | |
| | (SEQ ID NO: 23) | | | |
| | SKHSLSQ | 1/14 | 7.14 | Pept12 |
| | (SEQ ID NO: 5) | | | |
| | SMLDKSQ | 1/14 | 7.14 | |
| | (SEQ ID NO: 24) | | | |

TABLE 5
Primers used in the construction of the expression
vector for the peptide-mCherry fusion protein.
EcoRI site is underlined and lower-case text in-
dicates the coding portion for the peptide.

| Primer | Sequence |
|---|---|
| Peptide | TTT GAA TTC agt aag cat tct ctt tct cag |
| 12 | GGG GGG TCG GGG GGG TCG ATG GTG AGC AAG |
| Forward | GGC GAG G |
| | (SEQ ID NO: 25) |
| Peptide | TTT GAA TTC acg acg aag ttg cct aat tcg |
| 15 | GGG GGG TCG GGG GGG TCG ATG GTG AGC AAG |
| Forward | GGC GAG G |
| | (SEQ ID NO: 26) |
| Peptide | TTT GAA TTC gag acg ccg tct cgg gcg agg |
| 18 | GGG GGG TCG GGG GGG TCG ATG GTG AGC AAG |
| Forward | GGC GAG G |
| | (SEQ ID NO: 27) |
| Peptide | TTT GAA TTC aat aat agt ggg aag cag ctt |
| 22 | GGG GGG TCG GGG GGG TCG ATG GTG AGC AAG |
| Forward | GGC GAG G |
| | (SEQ ID NO: 28) |
| Reverse | GCT TGG CTG AAG CTT CTT GTA CAG CTC GTC |
| | CAT GCC |
| | (SEQ ID NO: 29) |
| Linker | GGG GGG TCG GGG GGG TCG |
| | (SEQ ID NO: 30) |

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention as described in the appended claims.

What is claimed is:

1. A chimeric Hemipteran insecticidal protein comprising an insect toxic portion and at least one target insect gut binding peptide or target insect gut binding peptide multimer portion, wherein said gut binding peptide is characterized by an amino acid sequence of peptide 12 (SEQ ID NO: 5), 15 (SEQ ID NO: 6), 18 (SEQ ID NO: 7), or 22 (SEQ ID NO: 8) and said Hemipteran toxic portion is Bt Cry1Ab (SEQ ID NO: 2) or Cry1Ba (SEQ ID NO: 1) or an insecticidal fragment thereof.

2. The chimeric insecticidal protein of claim 1, wherein the gut binding peptide or peptide multimer portion comprises an amino acid sequence selected from the group of one or more of Peptide 12 (S K H S L S Q) (SEQ ID NO: 5); Peptide 15 (T T K L P N S) (SEQ ID NO: 6); Peptide 18 (E T P S R A R) (SEQ ID NO: 7); and/or Peptide 22 (N N S G K Q L) (SEQ ID NO: 8).

3. The chimeric insecticidal protein of claim 1 wherein the insect toxic portion is a Cry1Ab (SEQ ID NO: 2), and/or Cry1Ba (SEQ ID NO: 1), or an insecticidal toxic fragment thereof, insecticidal toxin of Bacillus thuringiensis.

4. The chimeric insecticidal protein of claim 3, wherein the Cry1Ab (SEQ ID NO: 2) protein portion has the amino acid sequence set forth in SEQ ID NO:2 or an amino acid sequence with at least 85% amino acid sequence identity thereto.

5. The chimeric insecticidal protein of claim 1 wherein the insect toxic portion is produced by IBL Bacillus thuringiensis IBL-00200, IBL-00068, IBL-00365, IBL-00681, IBL-00048, IBL-00937, IBL-01306, IBL-01313, IBL-03792, and/or IBL-00829.

6. The chimeric insecticidal protein of claim 3, wherein the Cry1Ba (SEQ ID NO: 1) protein portion has the amino acid sequence set forth in SEQ ID NO:1 or an amino acid sequence with at least 85% amino acid sequence identity thereto.

7. The chimeric insecticidal protein of claim 3, wherein the Cry1Ab (SEQ ID NO: 2) or Cry1Ba (SEQ ID NO: 1), or insecticidal fragments thereof, protein portion includes a gut binding peptide as an N-terminal or C-terminal extension of the protein or within a modeling site.

8. A nucleic acid molecule comprising a sequence encoding the chimeric insecticidal protein of claim 1.

9. A nucleic acid molecule according to claim 8, wherein the gut binding peptide or peptide multimer portion comprises the amino acid sequence Peptide 12 (S K H S L S Q) (SEQ ID NO: 5); Peptide 15 (T T K L P N S) (SEQ ID NO: 6); Peptide 18 (E T P S R A R) (SEQ ID NO: 7); and/or Peptide 22 (N N S G K Q L) (SEQ ID NO: 8).

10. A construct comprising the sequence encoding the chimeric insecticidal protein of claim 1 is operably linked to a plant expressible promoter.

11. The construct according to claim 10, wherein the plant expressible promoter is a phloem-specific promoter.

12. The construct according to claim 8, wherein the plant expressible promoter is a constitutive promoter.

13. A vector comprising and expressing the construct of claim 8.

14. A transformed plant comprising the construct of claim 8.

15. A transformed plant according to claim 14, wherein the chimeric insecticidal protein is expressed in phloem tissue, leaf tissue, or root tissue of the plant.

16. A method of inhibiting plant damage by a target insect, said method comprising: providing a chimeric insecticidal protein of claim 1; and bringing a food source comprising the chimeric insecticidal protein into contact with a target insect under conditions that allow the target insect to ingest the food, whereby the chimeric insecticidal protein ingested by the insect inhibits feeding by or kills the target insect, resulting in reduced plant damage by the target insect.

17. The method of claim 16, wherein the target insect is a sap-sucking insect.

18. The method of claim 17, wherein the sap-sucking insect is an Asian citrus psyllid and wherein the chimeric insecticidal protein is expressed in phloem tissue of the plant.

19. The method of claim 17, wherein the food source is phloem tissue of a plant which contains and expresses the construct of claim 10 in the phloem tissue.

20. The method of claim 17, wherein the insect-transmitted bacterium causes huanglongbing disease

21. A method for inhibiting transmission of a plant pathogen, wherein said plant pathogen is spread from plant to plant by sap-sucking insects, comprising the step of providing a transgenic plant expressing the chimeric insecticidal protein of claim 1, whereby the sap-sucking insects are killed and transmission of the plant pathogen is reduced.

22. The method of claim 21, wherein the plant pathogen is bacteria associated with huanlongbing disease.

23. A host cell containing the vector of claim 13.

24. A host cell containing the construct of claim 10.

25. The host cell of claim 24, wherein said host cell is a plant cell.

26. The host cell of claim 25, wherein said plant cell is of a crop, ornamental or horticultural plant.

27. The host cell of claim 6, wherein said plant cell is of a citrus plant.

28. A virus or viral vector comprising the construct of claim 10.

29. The virus of claim 28 where said virus is CTV.

30. An insecticidal composition with activity against Hemiptera comprising: an effective amount of Bt toxin Cry1Ba (SEQ ID NO: 1) and a carrier.

31. The composition of claim 30 further comprising Cry1Ab (SEQ ID NO: 2).

32. The composition of claim 1 comprising a peptide with 90% amino acid sequence identity or greater with SEQ. ID. NO: 1 or 2.

34. A method of inhibiting plant damage by a target insect, said method comprising: providing a Bacillus thuringiensis Cry1Ba (SEQ ID NO: 1), Cry1Ba (SEQ ID NO: 1) and Cry1Ab (SEQ ID NO: 2), insecticidal fragments thereof, or toxin produced by Bt isolate IBL-00200, IBL-00068, IBL-00365, IBL-00681, IBL-00048, IBL-00937, IBL-01306, IBL-01313, IBL-03792 and/or IBL-00829 and applying an effective amount of said protein to the insect pest, wherein the mortality of said insect increases.

35. An insecticidal Hemiptera-active toxin comprising a modified insecticidal toxin that specifically binds to a receptor in a sap-sucking insect gut, and a Bt toxin.

36. The chimeric insecticidal protein of claim 7, wherein the Cry1Ab modeling sites is in loop1-2α of Domain I; or loop 2 or 3 of Domain II; or a combination thereof.

37. The chimeric insecticidal protein of claim 7, wherein the Cry1Ba modeling sites is in loop1-2α of Domain I; loop 2 or 3 of Domain II; or β16 or β22 of Domain III; or a combination thereof.
